(19) United States
(12) Patent Application Publication
(10) Pub. No.: US 2024/0091233 A1
(43) Pub. Date: Mar. 21, 2024

(54) USE OF WEE1 KINASE INHIBITORS IN THE TREATMENT OF CANCER

(71) Applicant: Impact Therapeutics (Shanghai), Inc, Shanghai (CN)

(72) Inventors: Ye Edward TIAN, Shanghai (CN); Sui Xiong CAI, Shanghai (CN); Mingchuan GUO, Shanghai (CN); Chih-Yi HSIEH, Shanghai (CN); Rong WANG, Shanghai (CN); Ruiyu ZHOU, Shanghai (CN)

(21) Appl. No.: 18/549,778

(22) PCT Filed: Mar. 9, 2022
(86) PCT No.: PCT/CN2022/079924

§ 371 Date: Sep. 8, 2023

(30) Foreign Application Priority Data
Mar. 10, 2021 (CN) 202110263595.X

(51) Int. Cl. A61K 31/519; A61K 45/06; A61P 35/00; C12Q 1/6874; C12Q 1/6886

(52) U.S. Cl. CPC A61K 31/519; A61K 45/06; A61P 35/00; C12Q 1/6874; C12Q 1/6886; C12Q 2600/106; C12Q 2600/156

(57) ABSTRACT

Provided is a use of Wee1 kinase inhibitors in the treatment of cancer. In particular, provided is a use of Wee1 kinase inhibitors in the preparation of drugs for the treatment of cancers with Histone H3K27M mutation.

### FIELD OF THE DISCLOSURE

This disclosure is in the field of medicinal chemistry, and relates to the field of cancer treatment. In particular, the disclosure relates to the use of Wee1 kinase inhibitors in the treatment of cancer with Histone H3K27M mutation.

### BACKGROUND OF THE INVENTION

The process of proliferation and division of eukaryotic cell is called the cell cycle. A cell grows gradually from a newly divided cell to two daughter cells through the cell cycle. The cell cycle roughly includes 4 distinct phases, the G1 phase, the S phase, the G2 phase and the M phase. The G1 phase is also called the phase of growth, which is characterized by active cell metabolism, large scale of synthesis of protein, carbohydrate, lipid and RNA and cells rapid growth from newly divided young cells to mature cells. There are also some cells that do not go on the cell cycle after completing the G1 phase and stay in this phase, and for those cells it is called to stay in G0 phase. The S stage of cell cycle is mainly characterized by DNA synthesis and replication, and the change of chromosome from diploid to tetraploid. Then the cell enters the G2 phase. The characteristic of G2 phase is that the cell continues to grow, and the synthesized DNA is checked for damages and mutations. The damaged DNA is repaired to prepare the cell for mitosis. The last phase of the cell cycle is the M phase. In this phase, the cell divides from one mother cell into two identical daughter cells through mitosis, completing cell reproduction. During the whole process of the cell cycle, there are several cell cycle checkpoints including G1/S or G1 checkpoint and G2/M or G2 checkpoint. The main functions of these checkpoints are to check and ensure the accurate replication of genetic information (DNA) during the cell cycle and to determine whether the cell enters the next phase of the cell cycle.

Each cell cycle checkpoint is a system composed of multiple factors that works through complex mechanisms. For instance, the G2/M checkpoint uses a complex process to check for DNA damage. Of this process, an important kinase is Cdk1, which forms a complex with Cyclin-B1 (Nurse, P., Nature, 1990, 344:503-508). The activation and inactivation of Cdk1 play a vital role in the cell entering mitosis (M) from the G2 phase and the subsequent completion of mitosis The activity of Cdk1 is regulated by multiple mechanisms, including the binding with Cyclin A or Cyclin B, and phosphorylation and dephosphorylation. Wee1 kinase phosphorylates Cdk1 and inhibits its activity therefore delays the cell from entering mitosis.

Wee1 is a tyrosine kinase that inhibits the activity of Cdk1 by phosphorylating tyrosine 15 (Y15) on Cdk1 molecule (McGowan, C. H., et al., EMBO J, 1993, 12:75-85; Parker, L. L., et al., Science, 1992, 257:1955-1957). Accordingly, Wee1 is a key inhibitory regulator of Cdk1 activity and plays an important role in G2-M phase checkpoints (O'Connell, M. J., et al, EMBO J, 1997, 16:545-554). Loss or inactivation of Wee1 may result in premature entry into mitosis, leading to mitotic failure and cell death (Stumpff, J., et al, Curr Biol, 2004, 14:2143-2148). Some tumor cells have functional deficiency or loss in G1/S phase cell cycle checkpoint function and heavily rely on G2/M phase checkpoints to ensure the progress of cell growth and division (Sancar, A., et al, Annu Rev Biochem, 2004, 73:39-85). In cancer cells the function of G1/S checkpoint is often lost due to reasons such as mutations in p53. These cells are heavily relying on G2/M checkpoint function when DNA damage happens and sensitive to Wee1 function loss (Wang, Y., et al, Cancer Biol & Ther, 2004, 3:305-313).

Inhibition of Wee1 activity may selectively promote the death of cancer cells with defective cell cycle checkpoints, but have less effect on normal cells with normal cell cycle checkpoint function. Therefore, Wee1 kinase inhibitors may be used as targeted therapy for the treatment of cancers and other disorders that have cell cycle checkpoint defects.

In recent years, several Wee1 kinase inhibitors have been developed and reported including compounds disclosed in WO2007126122 (substituted 1,2-dihydro-3H-pyrazolo[3,4-d]pyrimidin-3-one compounds), WO2019028008, WO2019173082 and WO202021032 (substituted 1,2-dihydro-3H-pyrazolo[3,4-d]pyrimidin-3-one compounds), WO2015092431 and WO2018162932 (substituted 2,3-dihydropyrimido[4,5-d]pyrimidin-4(1H)-one compounds), WO2019037678 (substituted 1,2-dihydro-3H-pyrazolo[3,4-d]pyrimidin-3-one compounds), WO2020210377 and WO2020210383 (substituted heterocyclic compounds), WO2020210375, WO2020210380, WO2020210381 (substituted 1,2-dihydro-3H-pyrazolo[3,4-d]pyrimidin-3-one compounds), WO2018133829 (1,2-dihydro-3H-pyrazolo[3,4-d]pyrimidin-3-one compounds) and WO2019085933 (1,2-dihydro-3H-pyrazolo[3,4-d]pyrimidine-3-one macrocyclic compounds). Among them, Adavosertib (AZD1775) is the first Wee1 kinase inhibitor to enter the clinic and is currently in clinical phase II. Wee1 kinase inhibitors that are currently in clinical phase include ZN-c3 and Debio-0123, which are in clinical phase I. There are also a variety of Wee1 kinase inhibitors currently in the preclinical research stage, such as DN-1609, NUV-569 and the like.

WO2018090939 discloses the following compounds of Formula I or pharmaceutically acceptable salts or prodrugs thereof as Wee1 kinase inhibitors.

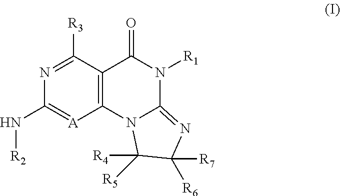

### Wherein, A is N or CR₁₅;

R₁ is hydrogen, optionally substituted C₁-C₈ alkyl, optionally substituted C₂-C₈ alkenyl, optionally substituted C₃-C₈ cycloalkyl, optionally substituted aryl, optionally substituted heterocyclic group or optionally substituted heteroaryl;
R₂ is optionally substituted carbocyclic group, optionally substituted heterocyclic group, optionally substituted aryl, or optionally substituted heteroaryl;
R₃-R₇ and R₁₅ are independently hydrogen, halo, optionally substituted amino, optionally substituted alkoxy, optionally substituted C₁-C₁₀ alkyl, haloalkyl, alkenyl, alkynyl, hydroxylalkyl, aminoalkyl, carboxylalkyl, nitro, cyano, acylamido, hydroxy, thiol, acyloxy, azido, carboxy, ethylenedioxo, hydroxylamido or optionally substituted alkylthiol.

WO2019011228 discloses the following compounds of Formula Il or pharmaceutically acceptable salts or prodrugs thereof as Wee1 kinase inhibitors.

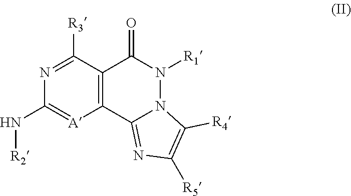

Wherein, A′ is N or CR₆′;
R₁′ is hydrogen, optionally substituted C₁-C₈ alkyl, optionally substituted C₂-C₈ alkenyl, optionally substituted C₃-C₈ cycloalkyl, optionally substituted aryl, optionally substituted heterocyclic group or optionally substituted heteroaryl;
R₂′ is optionally substituted carbocyclic group, optionally substituted heterocyclic group, optionally substituted aryl, or optionally substituted heteroaryl;
R₃′-R₆′ are independently hydrogen, halo, optionally substituted amino, optionally substituted alkoxy, optionally substituted C₁-C₁₀ alkyl (such as haloalkyl, hydroxylalkyl, aminoalkyl and carboxylalkyl), alkenyl, alkynyl, nitro, cyano, acylamido, hydroxy, thiol, acyloxy, azido, carboxy, ethylenedioxo, hydroxylamido or optionally substituted alkylthiol.

In addition, WO2021073491 discloses the following compounds of Formula III or pharmaceutically acceptable salts or prodrugs thereof as Wee1 kinase inhibitors.

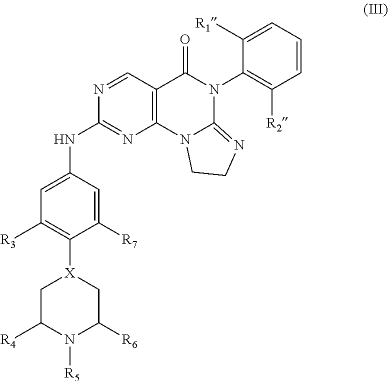

wherein, R₁″ and R₂″ are independently halo; R₃″ is halo, C₁₋₄ alkyl or C₁₋₄ alkoxy, R₄″ and R₆″ each are independently H or C₁₋₄ alkyl; R₅″ is H or C₁₋₄ alkyl; R₇″ is H, halo, C₁₋₄ alkyl or C₁₄₋ alkoxy; and X is CH or N;

Histone modification is a form of epigenetic mechanism and is involved in gene regulation. A lysine at 27 position of histone 3 (H3) is a methylation site. Its methylation such as H3K27me2 and H3K27me3 may play an important role in epigenetic mechanism. Histone H3.3 protein is encoded by H3F3A and H3F3B genes. A lysine mutation to methionine at position 27 of H3 (H3K27M) is frequently found in pediatric glioblastoma (GBM) especially in childhood diffuse intrinsic pontine glioma (DIPG) (Khuong-Quang, D.-A., et al., Acta Neuropathol, 2012, 124:439-47). The H3K27M mutation changes the methylation status of H3 and causes changes in the epigenetic landscape which globally affects gene expression (Harutyunyan, A. S., et al., Cell Rep, 2020, 33:108390-430). This H3K27M mutation seems to be a driving event of oncogenesis. H3K27M is found in up to 30% of pediatric GBM patients and 60% in DIPG (Wan, Y. C. E., et al., Curr Pharmacol Rep, 2018, 4:292-300).

Diffuse midline glioma (DMG) refers to high-grade brain gliomas that occur in midline structures such as the corpus callosum, third ventricle, thalamus, and brainstem. Due to the location of the disease and the characteristics of invasive growth, the disease is difficult to treat and the prognosis is extremely poor. Due to the unique mutation of H3K27M, DMG was separately classified into a new type in the 2016 WHO classification of central nervous system tumors. The diagnosis needs to satisfy with the following criteria, the tumor is located in the midline of the central nervous system; it shows diffuse growth; and it has H3K27M mutation. Regardless of whether its histological morphology meets high-level features, the tumor will be diagnosed as grade IV (Louis, D. N., et al., Acta Neuropathol, 2016, 131:803-20). The H3K27M mutation is rare in adult gliomas and other types of tumors, but it is common in childhood midline gliomas. Studies have shown that in DMG the H3K27M mutation significantly correlates with the patient's prognosis. For instance, for patients of thalamic glioma diagnosed at 8 years of age with wildtype H3, the overall survival (OS) is about 11 years, whereas for patients of thalamic glioma diagnosed at 10 years of age with H3K27M mutation, the OS (overall survival) is only 1.8 years. The 5-year survival rate for children with thalamic glioma with H3K27M mutation is 6.3%, whereas for children with wildtype H3 it is 68.8% (Ryall, S., et al., Acta Neuropathol Commun, 2016, 4:93-102). Regardless it is high-grade (HGG) or low-grade glioma (LGG), glioma patients with H3K27M mutation have a shorter survival time.

The current treatment of DMG is a combination of surgery and adjuvant radiotherapy and chemotherapy. The survival prognosis of children with DMG is still very poor. For patients with DMG, especially with H3K27M mutations, there is an urgent need to find effective treatment.

The present disclosure found that Wee1 kinase inhibitor has good efficacy in a mouse model of human DMG with H3K27M mutation. Significant inhibitory effects of Wee1 kinase inhibitor in the model have been observed, suggesting a potential treatment for DMG cancer patients with H3K27M mutation

### SUMMARY OF THE DISCLOSURE

The disclosure finds that the growth of DMG cancer cells with H3K27M mutation can be effectively inhibited by inhibiting Wee1 activity, which is of great significance towards the treatment of the disease.

The disclosure provides Wee1 kinase inhibitors for use in a method for the treatment or prevention of a cancer with H3K27M mutation, especially in the treatment or prevention of DMG with H3K27M mutation.

The disclosure also provides use of Wee1 kinase inhibitors in the preparation of a medicament for the treatment or prevention of a cancer with H3K27M mutation.

The disclosure also provides a method for the treatment or prevention of a cancer with H3K27M mutation, comprising administering to a subject in need thereof an effective amount of a Wee1 kinase inhibitor or its pharmaceutical composition.

Useful Wee1 kinase inhibitors include but are not limited to those described herein, in particular, including but not limited to AZD1775, Zc-03, Debio-0123, DN-1609, NUV-569, and compounds disclosed in WO2007126122 (substituted 1,2-dihydro-3H-pyrazolo[3,4-d]pyrimidin-3-one compounds), WO2019028008, WO2019173082 and WO202021032 (substituted 1,2-dihydro-3H-pyrazolo[3,4-d]pyrimidin-3-one compounds), WO2015092431 and WO2018162932 (substituted 2,3-dihydropyrimido[4,5-d]pyrimidin-4(1H)-one compounds), WO2019037678 (substituted 1,2-dihydro-3H-pyrazolo[3,4-d]pyrimidin-3-one compounds), WO2020210377 and WO2020210383 (substituted heterocyclic compounds), WO2020210375, WO2020210380, WO2020210381 (substituted 1,2-dihydro-3H-pyrazolo[3,4-d]pyrimidin-3-one compounds), WO2018133829 (1,2-dihydro-3H-pyrazolo[3,4-d]pyrimidin-3-one compounds), WO2019085933 (1,2-dihydro-3H-pyrazolo[3,4-d]pyrimidine-3-one macrocyclic compounds), WO2018090939, WO2019011228 and WO2021073491.

In one or more embodiments, the method comprises administering an effective amount of a compound of Formula I, II or III, or a stereoisomer, a pharmaceutically acceptable salt or a prodrug thereof as described herein.

In one or more embodiments, the cancer with H3K27M mutation is glioma with H3K27M mutation, including but not limited to glioblastoma, childhood diffuse pontine glioma and diffuse midline glioma.

### DESCRIPTION OF DRAWING

FIG. 1: Correlation analyses demonstrate that H3K27M significantly related to compound response

FIG. 2: Unsupervised clustering of single-cell expression data. Left panel: 2D representation of sample correlations by tSNE dimensionality reduction, color-coded by tumor sample; Right panel: identical 2D color-coded for 18 cell populations.

FIG. 3: Pair-wise correlation analyses between the expression profiles (top panel) or CNV (bottom panel) of 3 samples (rows, columns) of each model. For the gene expression profiling, top 3000 variated genes of each cell in the indicated sample were selected to calculate the averaged medium.

FIG. 4: Characterization of cell types and cell cycle of tumor cells in scRNA-seq. The cell type and cell cycle of each cell are identified by the distribution difference of the expression of gene sets compared to the expression of the rest of genes. The heat maps are based on the expression of top 5 highly expressed genes of each cell type. Each column represents one cell.

FIG. 5: The percentage of H3K27M cells are plotted based on the cell type and the status of cell cycle. X-axis represent pre-treatment (M574, M309), 28 days vehicle (M574V, M309V), and 28 days compound-treated (MS74T, M309T). The ratio of Y-axis is calculated by the number of indicated cell type divided by the total cell number of the indicated sample.

### DETAILED DESCRIPTION OF THE DISCLOSURE

Cancer cells (or tumor cells) described herein are generally characterized by abnormal proliferation relative to normal cells and the formation of clusters or tumors in individuals suffering from cancer.

Cancers with H3K27M mutation described herein may include any type of solid tumors and malignant lymphomas, especially gliomas, including but not limited to glioblastoma, childhood diffuse pontine glioma and diffuse midline glioma. In preferred embodiments, the cancer with H3K27M mutation is diffuse midline glioma. Cancer can be familial or sporadic. In some embodiments, the cancers with H3K27M mutation may further contain TP53 mutation. Additionally or alternatively, the cancers with H3K27M mutation may further contain one or more mutations selected from the group consisting of ATRX deletion or mutation, PDGFRA or KIT or KDR amplification, BRCA2 deletion, and SRC amplification. In some embodiments, the diffuse midline glioma as described herein is a diffuse midline glioma with H3K27M mutation and TP53 mutation, and optionally one or more mutations selected from the group consisting of ATRX deletion or mutation, PDGFRA or KIT or KDR amplification, BRCA2 deletion, and SRC amplification. In some embodiments, the diffuse midline glioma as described herein contains the H3K27M mutation in oligodendrocyte precursor cells.

A sample obtained from an individual may be a tissue sample containing one or more cells, such as the cancer tissue described above, or a non-cancer tissue biopsy sample used as a control.

An individual may have a cancer, and the sample may be a cancer cell sample from a tumor biopsy.

The method of the disclosure can be used to evaluate an individual suffering from a cancer so as to determine, for example, the therapeutic duration of an action. Methods for evaluating an individual with a cancer may include identifying the cancer cells obtained from the individual as having H3K27M mutations and providing Wee1 kinase inhibitors suitable for administration to the individual.

In some embodiments, the cancer cell can have H3K27M mutation phenotype.

Individuals may have cancer with H3K27M mutation. The methods and means of the present disclosure are particularly applicable to such individuals.

An individual may be, for example, a heterozygote with H3K27M mutation or polymorphism.

A method for treating cancer in an individual may include administering a Wee1 kinase inhibitor to the individual, wherein the individual is a heterozygote with H3K27M mutation or polymorphism.

Wee1 kinase inhibitor can be used to prepare a drug for treating cancer in an individual, wherein the individual is a heterozygote with H3K27M mutation or polymorphism.

Wee1 kinase inhibitors suitable for the method can be any compound or entity that inhibits, reduces or eliminates Wee1 kinase, such as small organic molecules, peptides or nucleic acids.

Examples of compounds that are known as Wee1 kinase inhibitors and can be used according to the present disclosure include, but are not limited to: AZD1775, Ze-03, Debio-0123, DN-1609, NUV-569, and compounds disclosed in WO2007126122, WO2019028008, WO2019173082, WO202021032, WO2015092431, WO2018162932, WO2019037678. WO2020210377, WO2020210383, WO2020210375, WO2020210380, WO2020210381, WO2018133829, WO2019085933, WO2018090939, WO2019011228 and WO2021073491.

The method of the present disclosure can also be used to assess cancer in an individual.

Genetic testing methods are used to detect H3K27M mutations in cancer, and after confirmation of H3K27M mutations, Wee1 kinase inhibitors can be used for treatment.

The method for evaluating H3K27M mutation in a cancer may also include: contacting a Wee1 kinase inhibitor with a cancer cell sample obtained from an individual suffering from the cancer, and determining the number of dead cells in the sample relative to a control sample.

Compared with control cells, the increase of the sensitivity of the Wee1 kinase inhibitor in the sample cells indicates that the cancer cells may have H3K27M mutations, such as reduction or loss of H3K27M expression or activity.

In preferred embodiments, the Wee1 kinase inhibitor is an inhibitor disclosed in WO2018090939, which is incorporated herein by reference in its entirety. More specifically, the Wee1 kinase inhibitor is a compound represented by Formula I.

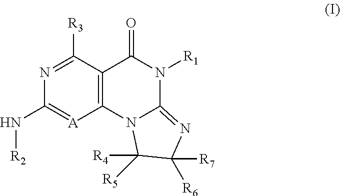

or a pharmaceutically acceptable salt or a prodrug thereof, wherein A is N or CR₁₅;
R₁ is hydrogen, optionally substituted C₁-C₈ alkyl, optionally substituted C₂-C₈ alkenyl, optionally substituted C₃-C₈ cycloalkyl, optionally substituted aryl, optionally substituted heterocyclic group or optionally substituted heteroaryl;
R₂ is an optionally substituted carbocyclic group, an optionally substituted heterocyclic group, an optionally substituted aryl, or an optionally substituted heteroaryl;
R₃-R₇ and R₁₅ are independently hydrogen, halo, optionally substituted amino, optionally substituted alkoxy, optionally substituted C₁-C₁₀ alkyl, haloalkyl, alkenyl, alkynyl, hydroxylalkyl, aminoalkyl, carboxylalkyl, nitro, cyano, acylamido, hydroxy, thiol, acyloxy, azido, carboxy, ethylenedioxo, hydroxylamido or optionally substituted alkylthiol.

In one or more embodiment, A is N.

In one or more of the foregoing embodiments. R₁ and R₂ are optionally substituted aryl.

In one or more of the foregoing embodiments, R₃-R₇ are each independently H, halo, or C₁-C₆ alkyl.

In one or more of the foregoing embodiments, R₁₅ is H or C₁-C₆ alkyl.

In one or more of the foregoing embodiments, the substituents on R₁ are selected from any one, two or three of the following groups: halo, C₁-C₆ alkyl, C₁-C₆ alkoxy, and halo C₁-C₆ alkyl.

In one or more of the foregoing embodiments, R₁ is selected from: H, C₁-C₈ alkyl, C₂-C₈ alkenyl, C₃-C₈ cycloalkyl, heteroaryl, and aryl which is optionally substituted by 1-4 groups selected from halo, C₁-C₆ alkyl, C₁-C₆ alkoxy, and halo C₁-C₆ alkyl.

In one or more of the foregoing embodiments, R₁ is selected from phenyl which is optionally substituted by 1-4 groups selected from halo, C₁-C₆ alkyl, C₁-C₆ alkoxy, and halo C₁-C₆ alkyl; in some embodiments, the number of substituents is 2; in some embodiments, at least one substituent is in the ortho position, in some embodiments, at least one substituent is halo; in some embodiments, the number of substituents on the phenyl is 2, both of which are located adjacent to each other, and wherein at least one is halo.

In one or more of the foregoing embodiments, R₁ is selected from optionally substituted pyridyl, pyrimidyl, thiophenyl, furanyl, pyrrolyl and imidazolyl

In one or more of the foregoing embodiments, R₁ is selected from H, optionally substituted C₁-C₈ alkyl, C₃-C₈ cycloalkyl, and C₂-C₈ alkenyl.

In one or more of the foregoing embodiments, the substituents on R₂ are selected from any one, two, three or four of the following groups: optionally substituted C₁-C₆ alkyl, optionally substituted C₁-C₆ acyl, optionally substituted heterocyclic group, halo, optionally substituted oxy group, nitro, and optionally substituted C₁-C₆ alkylamino; preferably, the substituents on these substituted group may be 1-4 groups selected from the following group: C₁-C₆ alkyl, C₁-C₆ acyl, a heterocyclic group optionally substituted by 1-4 of C₁-C₆ alkyl, halo, —NR₂R_{b} and hydroxy, wherein Rₐ and R_{b} are each independently H and C₁-C₆ alkyl; preferably, the heterocyclic group is selected from piperazinyl, piperidinyl, morpholinyl, and 1,4-diazacycloheptyl

In one or more of the foregoing embodiments, the substituents on R₂ are selected from any one, two, three or four of the following groups: optionally substituted piperazinyl, optionally substituted piperazinyl-C₁-C₄ alkyl, optionally substituted piperidinyl, imidazolyl, optionally substituted 1,4-diazacycloheptyl, C₁-C₆ alkyl, C₁-C₆ acyl, optionally substituted morpholinyl, morpholinyl-C₁-C₅ alkyl, halo, halo C₁-C₆ alkyl, optionally substituted C₁-C₆ alkoxy, optionally substituted hydroxy C₁-C₆ alkyl, optionally substituted amino C₁-C₆ alkyl, optionally substituted piperidinylamino, optionally substituted C₁-C₆ alkyl amino, optionally substituted heterocyclic alkyl-O— and nitro; preferably, the substituents on the optionally substituted group may be 1-4 groups selected from the following groups: C₁-C₆ alkyl, C₁-C₆ acyl, halo, —NRₐR_{b} and C-C₆ alkyl substituted by hydroxy, wherein Rₐ and R_{b} are each independently H and C₁-C₆ alkyl.

In one or more of the foregoing embodiments, the optionally substituted piperazinyl is the piperazinlyl which can be substituted by 1, 2 or 3 groups selected from: C₁-C₆ alkyl, hydroxy C₁-C₆ alkyl, and C₁-C₆ acyl.

In one or more of the foregoing embodiments, the piperazine group has at least one substituent at the para-position, and optionally, one or two substituents at the meta-position.

In one or more of the foregoing embodiments, the optionally substituted piperidinyl is the piperidinyl which can be substituted by 1 group selected from C₁-C₆ alkyl and C₁-C₆ alkyl amino.

In one or more of the foregoing embodiments, the optionally substituted morpholinyl is the morpholinyl which can be substituted by 1 or 2 groups selected from C₁-C₆ alkyl.

In one or more of the foregoing embodiments, R₂ is selected from optionally substituted phenyl, pyridyl, piperazinyl, tetrahydroisoquinolinyl, 2′,3′-dihydro-1′H-spiro[cyclopropane-1,4′-isoquinolin]-7-yl and 4,5,6,7-tetrahydropyrazolo[1,5-a]pyrazin-2-yl.

In one or more of the foregoing embodiments, R₂ is selected from phenyl substituted by optionally substituted piperazinyl, phenyl substituted by optionally substituted pyridinyl, and tetrahydroisoquinolinyl optionally substituted by one to three C₁-C₆ alkyls or halos.

In one or more of the foregoing embodiments, the piperazinyl is optionally substituted by one to three groups selected from C₁-C₆ alkyl, hydroxy C₁-C₆ alkyl and C₁-C₆ acyl.

In one or more of the foregoing embodiments, the piperidinyl is optionally substituted by 1 group selected from C₁-C₆ alkyl and C₁-C₆ alkyl amino.

In one or more of the foregoing embodiments, R₄ and R₅ are each independently H, C₁-C₆ alkyl and halo, preferably, both H.

In one or more of the foregoing embodiments, R₆ and R₇ are each independently H, C₁-C₆ alkyl and halo, preferably, both H.

In one or more embodiments, preferred compounds of Formula I include, without limitation:
6-(2-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-8,9-dihydroimidazo[1,2-a]pyrimido[5,4-e]pyrimidin-5(6H)-one;
4-(2-chlorophenyl)-8-((4-(4-methylpiperazin-1-yl)phenyl)amino)-1,2-dihydroimidazo[1,2-a]pyrido[3,4-e]pyrimidin-5(4H)-one;
6-(2,6-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-isopropylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-acetylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2-chloro-6-fluorophenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2-chloro-6-fluorophenyl)-2-((2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2-chloro-6-fluorophenyl)-2-((2′-acetyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2′-methyl-2′,3′-dihydro-1′H-spiro[cyclopropane-1,4′-isoquinolin]-7-yl)amino)-8,9-dihydroimidazo[1,2-a]pyrimido[5,4-e]pyrimidin-5(6H)-one;
6-(2,6-dimethylphenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dimethylphenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dimethylphenyl)-2-((2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-isopropyl-2-((4-(4-methylpiperazin-1-yl)phenyl)amino)-8,9-dihydroimidazo[1,2-a]pyrimido[5,4-e]pyrimidin-5(6H)-one;
6-(tert-butyl)-2-((4-(4-methylpiperazin-1-yl)phenyl)amino)-8,9-dihydroimidazo[1,2-a]pyrimido[5,4-e]pyrimidin-5(6H)-one;
6-cyclopropyl-2-((4-(4-methylpiperazin-1-yl)phenyl)amino)-8,9-dihydroimidazo[1,2-a]pyrimido[5,4-e]pyrimidin-5(6H)-one;
6-cyclohexyl-2-((4-(4-methylpiperazin-1-yl)phenyl)amino)-8,9-dihydroimidazo[1,2-a]pyrimido[5,4-e]pyrimidin-5(6H)-one;
6-allyl-2-((4-(4-methylpiperazin-1-yl)phenyl)amino)-8,9-dihydroimidazo[1,2-a]pyrimido[5,4-e]pyrimidin-5(6H)-one;
6-(thiophen-2-yl)-2-((4-(4-methylpiperazin-1-yl)phenyl)amino)-8,9-dihydroimidazo[1,2-a]pyrimido[5,4-e]pyrimidin-5(6H)-one;
6-(furan-2-yl)-2-((4-(4-methylpiperazin-1-yl)phenyl)amino)-8,9-dihydroimidazo[1,2-a]pyrimido[5,4-e]pyrimidin-5(6H)-one;
6-(1H-pyrrol-2-yl)-2-((4-(4-methylpiperazin-1-yl)phenyl)amino)-8,9-dihydroimidazo[1,2-a]pyrimido[5,4-e]pyrimidin-5(6H)-one;
6-(1H-Imidazol-5-yl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-8,8-dimethyl-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-9,9-dimethyl-8,9-dihydroimidazo[1,2-a]pyrimido[5,4-e]pyrimidin-5(6H)-one;
6-(2-chloro-6-fluorophenyl)-2-((4-((2S,6R)-2,6-dimethylmorpholino)phenyl)amino)-8,9-dihydroimidazo[1,2-a]pyrimido[5,4-e]pyrimidin-5(6H)-one;
6-(2-chloro-6-fluorophenyl)-2-((4-(morpholinomethyl)phenyl)amino)-8,9-dihydroimidazo[1,2-a]pyrimido[5,4-e]pyrimidin-5(6H)-one;
6-(2-chloro-6-fluorophenyl)-2-((4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-fluoro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-fluoro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-chloro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-2-(trifluoromethyl)phenyl)amino)-8,9-dihydroimidazo[1,2-a]pyrimido[5,4-e]pyrimidin-5(6H)-one;
6-(2-chloro-6-fluorophenyl)-2-((2-trifluoromethyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-3-(trifluoromethyl)phenyl)amino)-8,9-dihydroimidazo[1,2-a]pyrimido[5,4-e]pyrimidin-5(6H)-one;
6-(2-chloro-6-fluorophenyl)-2-((3-trifluoromethyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-isopropylpiperazin-1-yl)-3-methylphenyl)amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)-3-methylphenyl)amino)-8,9-dihydroimidazo[1,2-a]pyrimido[5,4-e]pyrimidin-5(6H)-one;
6-(2-chloro-6-fluorophenyl)-2-((3-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-3-nitrophenyl)amino)-8,9-dihydroimidazo[1,2-a]pyrimido[5,4-e]pyrimidin-5(6H)-one;
6-(2-chloro-6-fluorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((6-(4-methylpiperazin-1-yl)pyridin-3-yl)amino)-8,9-dihydroimidazo[1,2-a]pyrimido[5,4-e]pyrimidin-5(6H)-one;
6-(2-chloro-6-fluorophenyl)-2-((2′-isopropyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2-chloro-6-fluorophenyl)-2-((5-methyl-4,5,6,7-tetrahydropyrazolo[1,5-a]pyrazin-2-yl)amino)-8,9-dihydroimidazo[1,2-a]pyrimido[5,4-e]pyrimidin-5(6H)-one;
6-(2,6-difluorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-(trifluoromethyl)phenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-isopropylpiperazin-1-yl)phenyl)amino)-8,9-dihydroimidazo[1,2-a]pyrimido[5,4-e]pyrimidin-S(6H)-one;
6-(2,6-dichlorophenyl)-2-((4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-((3S,5R)-4-isopropyl-3,5-dimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-chloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-trifluoromethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-methylpiperazin-1-yl)-3-(trifluoromethyl)phenyl)amino)-8,9-dihydroimidazo[1,2-a]pyrimido[5,4-e]pyrimidin-5(6H)-one;
6-(2,6-dichlorophenyl)-2-((3-trifluoromethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-(3-methyl-(4-(4-isopropylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5R)-4-isopropyl-3,5-dimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((5-methyl-4,5,6,7-tetrahydropyrazolo[1,5-a]pyrazin-2-yl)amino)-8,9-dihydroimidazo[1,2-a]pyrimido[5,4-e]pyrimidin-5(6H)-one;
6-(2-chloro-6-trifluoromethylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methoxyphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dimethylphenyl)-2-((4-(4-methylpiperazin-1-yl)phenyl)amino)-8,9-dihydroimidazo[1,2-a]pyrimido[5,4-e]pyrimidin-5(6H)-one;
6-(2,6-dimethylphenyl)-2-((4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(4-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(3-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(2,4-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-4-fluorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-3-fluorophenyl)-2-((4-(4-methylpiperazin-1-yl)phenyl)amino)-8,9-dihydroimidazo[1,2-a]pyrimido[5,4-e]pyrimidin-5(6H)-one;
6-(2,5-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2,3-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(pyrimidin-2-yl)-2-((4-(4-methylpiperazin-1-yl)phenyl)amino)-8,9-dihydroimidazo[1,2-a]pyrimido[5,4-e]pyrimidin-5(6H)-one;
2-((4-(4-methylpiperazin-1-yl)phenyl)amino)-8,9-dihydroimidazo[1,2-a]pyrimido[5,4-e]pyrimidin-5(6H)-one;
6-cyclobutyl-2-((4-(4-methylpiperazin-1-yl)phenyl)amino)-8,9-dihydroimidazo[1,2-a]pyrimido[5,4-e]pyrimidin-5(6H)-one;
6-cyclopentyl-2-((4-(4-methylpiperazin-1-yl)phenyl)amino)-8,9-dihydroimidazo[1,2-a]pyrimido[5,4-e]pyrimidin-5(6H)-one;
6-phenyl-2-((4-(4-methylpiperazin-1-yl)phenyl)amino)-8,9-dihydroimidazo[1,2-a]pyrimido[5,4-e]pyrimidin-5(6H)-one;
6-(pyridin-2-yl)-2-((4-(4-methylpiperazin-1-yl)phenyl)amino)-8,9-dihydroimidazo[1,2-a]pyrimido[5,4-e]pyrimidin-5(6H)-one;
6-(pyridin-3-yl)-2-((4-(4-methylpiperazin-1-yl)phenyl)amino)-8,9-dihydroimidazo[1,2-a]pyrimido[5,4-e]pyrimidin-5(6H)-one;
6-(pyridin-4-yl)-2-((4-(4-methylpiperazin-1-yl)phenyl)amino)-8,9-dihydroimidazo[1,2-a]pyrimido[5,4-e]pyrimidin-5(6H)-one;
6-(2,6-difluorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2,6-difluorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(1H-imidazol-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methyl-1,4-diazacyclohept-1-yl)phenyl)amino)-8,9-dihydroimidazo[1,2-a]pyrimido[5,4-e]pyrimidin-5(6H)-one;
6-(2-chloro-6-fluorophenyl)-2-((4-((4-methylpipcrazin-1-yl)methyl)phenyl)amino)-8,9-dihydroimidazo[1,2-a]pyrimido[5,4-e]pyrimidin-5(6H)-one;
6-(2-chloro-6-fluorophenyl)-2-((4-(2-(dimethylamino)ethoxy)phenyl)amino)-8,9-dihydroimidazo[1,2-a]pyrimido[5,4-e]pyrimidin-5(6H)-one;
6-(2-chloro-6-fluorophenyl)-2-((4-(3-(dimethylamino)propoxy)phenyl)amino)-8,9-dihydroimidazo[1,2-a]pyrimido[5,4-e]pyrimidin-5(6H)-one;
6-(2-chloro-6-fluorophenyl)-2-((4-((1-methylpiperidin-4-yl)oxy)phenyl)amino)-8,9-dihydroimidazo[1,2-a]pyrimido[5,4-e]pyrimidin-5(6H)-one;
6-(2-chloro-6-fluorophenyl)-2-((4-((2-(dimethylamino)ethyl)amino)phenyl)amino)-8,9-dihydroimidazo[1,2-a]pyrimido[5,4-e]pyrimidin-5(6H)-one;
6-(2-chloro-6-fluorophenyl)-2-((4-((2-(dimethylamino)ethyl)(methyl)amino)phenyl)amino)-8,9-dihydroimidazo[1,2-a]pyrimido[5,4-e]pyrimidin-5(6H)-one;
6-(2-chloro-6-fluorophenyl)-2-((4-((3-(dimethylamino)propyl)amino)phenyl)amino)-8,9-dihydroimidazo[1,2-a]pyrimido[5,4-e]pyrimidin-5(6H)-one;
6-(2-chloro-6-fluorophenyl)-2-((4-((3-(dimethylamino)propyl)(methyl)amino)phenyl)amino)-8,9-dihydroimidazo[1,2-a]pyrimido[5,4-e]pyrimidin-5(6H)-one;
6-(2-chloro-6-fluorophenyl)-2-((4-((1-methylpiperidin-4-yl)amino)phenyl)amino)-8,9-dihydroimidazo[1,2-a]pyrimido[5,4-e]pyrimidin-5(6H)-one;
6-(2-chloro-6-fluorophenyl)-2-((4-(methyl(1-methylpiperidin-4-yl)amino)phenyl)amino)-8,9-dihydroimidazo[1,2-a]pyrimido[5,4-e]pyrimidin-5(6H)-one;
6-(2-chloro-6-fluorophenyl)-2-((4-(1-methylpiperidin-4-yl)phenyl)amino)-8,9-dihydroimidazo[1,2-a]pyrimido[5,4-e]pyrimidin-5(6H)-one;
6-(2-chloro-6-fluorophenyl)-2-((4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-bromo-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((5-(4-methylpiperazin-1-yl)pyrazin-2-yl)amino)-8,9-dihydroimidazo[1,2-a]pyrimido[5,4-e]pyrimidin-S(6H)-one;
6-(2-chloro-6-fluorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-bromo-5-methoxy-4-(4-methylpiperazin-1-yl)phenyl)amino)-8,9-dibydroimidazo[1,2-a]pyrimido[5,4-e]pyrimidin-5(6H)-one;
6-(2-chloro-6-fluorophenyl)-2-((3-methyl-5-trifluoromethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methoxy-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((5-chloro-2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,5-dimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((5-chloro-2,4,4-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,4,4,5-tetramethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((5′-chloro-2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((2′,5′-dimethyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((3,5-dichloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-bromo-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3,5-dichloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methoxy-4-(4-(dimethylamino)piperidin-1-yl)phenyl)amino)-8,9-dihydroimidazo[1.2-a]pyrimido[5,4-e]pyrimidin-5(6H)-one;
6-(2,6-dichlorophenyl)-2-((3-bromo-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-S-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-trifluoromethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-chloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-bromo-6-chlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-bromo-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-bromo-5-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-fluoro-5-methyl-4-(4-(dimethylamino)piperidin-1-yl)phenyl)amino)-8,9-dibydroimidazo[1,2-a]pyrimido[5,4-e]pyrimidin-5(6H)-one;
6-(2-chloro-6-methylphenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-4-(piperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-(hydroxymethyl)-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-(hydroxymethyl)-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-(hydroxymethyl)-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-(2-hydroxyethyl)piperazin-1-yl)phenyl)amino)-8,9-dihydroimidazo[1,2-a]pyrimido[5,4-e]pyrimidin-5(6H)-one;
6-(2,6-dichlorophenyl)-2-((4-morpholinophenyl)amino)-8,9-dihydroimidazo[1,2-a]pyrimido[5,4-e]pyrimidin-5(6H)-one;
6-(2,6-dichlorophenyl)-2-((3-((methylamino)methyl)-4-morpholinophenyl)amino)-8,9-dihydroimidazo[1,2-a]pyrimido[5,4-e]pyrimidin-5(6H)-one;
6-(2-bromo-6-chlorophenyl)-2-((3-chloro-4-(4-(dimethylamino)piperidin-1-yl)phenyl)amino)-8,9-dihydroimidazo[1,2-a]pyrimido[5.4-e]pyrimidin-5(6H)-one;
6-(2,6-dichlorophenyl)-2-((3-chloro-4-(4-methylpiperazin-1-yl)phenyl)amino)-8,9-dihydroimidazo[1,2-a]pyrimido[5,4-e]pyrimidin-5(6H)-one dihydrochloride;
   or a pharmaceutically acceptable salt or a prodrug thereof.

In particularly preferred embodiments, the Wee1 kinase inhibitor is 6-(2,6-dichlorophenyl)-2-((3-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one, or a pharmaceutically acceptable salt or a prodrug thereof.

In preferred embodiments, the Wee1 kinase inhibitor suitable for the present disclosure is a compound disclosed in WO2019011228, or a pharmaceutically acceptable salt or prodrug thereof, the entire content of which is incorporated herein by reference. More specifically, the Wee1 kinase inhibitor suitable for the present disclosure is a compound represented by the following Formula II:

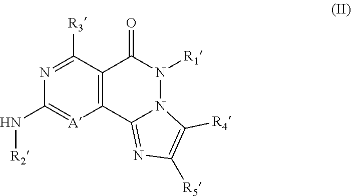

or a pharmaceutically acceptable salt or a prodrug thereof, wherein A′ is N or CR₆;
R₁′ is hydrogen, optionally substituted C₁-C₈ alkyl, optionally substituted C₂-C₈ alkenyl, optionally substituted C₃-C₈ cycloalkyl, optionally substituted aryl, optionally substituted heterocyclic group or optionally substituted heteroaryl;
R₂′ is an optionally substituted carbocyclic group, an optionally substituted heterocyclic group, an optionally substituted aryl, or an optionally substituted heteroaryl;
R₃′-R₆′ are independently hydrogen, halo, optionally substituted amino, optionally substituted alkoxy, optionally substituted C₁-C₁₀ alkyl (such as haloalkyl, hydroxylalkyl, aminoalkyl and carboxylalkyl), alkenyl, alkynyl, nitro, cyano, acylamido, hydroxy, thiol, acyloxy, azido, carboxy, ethylenedioxo, hydroxylamido or optionally substituted alkylthiol.

In one or more embodiments, A′is N.

In one or more of the foregoing embodiments, R₁′ and R₂′ are each optionally substituted aryl.

In one or more of the foregoing embodiments, R₃′ is N.

In one or more of the foregoing embodiments, R₄′ and R₅′ are each H and optionally substituted C₁-C₆ alkyl.

In one or more of the foregoing embodiments, R₄′ is H or unsubstituted C₁-C₆ alkyl.

In one or more of the foregoing embodiments, R₅′ is H or C-C₆ alkyl optionally substituted by hydroxy, such as hydroxy C₁-C₆ alkyl.

In one or more of the foregoing embodiments, R₆′ is H.

In one or more of the foregoing embodiments, the substituents on R₁′ are selected from any one, two, three or four of the following groups: halo, C₁-C₆ alkyl, C₁-C₆ alkoxy, and halo C₁-C₆ alkyl.

In one or more of the foregoing embodiments, R₁′ is selected from: C₂-C₈ alkenyl, and phenyl which is optionally substituted by one to four substituents selected from halo and C₁-C₆ alkyl.

In one or more of the foregoing embodiments, R₁′ is selected from phenyl which is optionally substituted by one to four substituents selected from halo and C₁-C₆ alkyl; in some embodiments, the number of substituents is 2; in some embodiments, at least one substituent is in the ortho position; in some embodiments, at least one substituent is halo; in some embodiments, the number of substituents on the phenyl is 2, both of which are located adjacent to each other, and wherein at least one is halo.

In one or more of the foregoing embodiments, R₁′ is selected from optionally substituted C₂-C₈ alkenyl.

In one or more of the foregoing embodiments, the substituents on R₂′ are selected from any one, two, three or four of the following groups: optionally substituted C₁-C₆ alkyl, optionally substituted oxy group, halo, and optionally substituted heterocyclic group; preferably, the substituents on these optionally substituted group may be one to four groups selected from the following groups: C₁-C₆ alkyl and —NRₐR_{b}, wherein Rₐ and R_{b} are each independently H and C₁-C₆ alkyl; preferably, the heterocyclic group is selected from piperazinyl and piperidinyl.

In one or more of the foregoing embodiments, the substituents on R₂′ are selected from any one, two, three or four of the following groups: optionally substituted piperazinyl, optionally substituted piperidinyl, C₁-C₆ alkyl, halo, and C₁-C₆ alkoxy; preferably, the substituents on the optionally substituted group may be one to four groups selected from the following groups: C₁-C₆ alkyl and —NRₐR_{b}, wherein Rₐ and R_{b} are each independently H and C₁-C₆ alkyl.

In one or more of the foregoing embodiments, the optionally substituted piperazinyl is piperazinlyl which can be substituted by 1, 2 or 3 groups selected from: C₁-C₆ alkyl.

In one or more of the foregoing embodiments, the piperazine group has at least one substituent at the para-position, and optionally, one or two substituents at the meta-position.

In one or more of the foregoing embodiments, the optionally substituted piperidinyl is piperidinyl which can be substituted by 1 group selected from C₁-C₆ alkyl and —NRₐR_{b}, wherein Rₐ and R_{b} are each independently H and C₁-C₆ alkyl.

In one or more of the foregoing embodiments, R₂′ is selected from optionally substituted phenyl and optionally substituted tetrahydroisoquinolinyl

In one or more of the foregoing embodiments. R₂′ is selected from phenyl substituted by optionally substituted piperazinyl, phenyl substituted by optionally substituted pyridinyl, and tetrahydroisoquinolinyl optionally substituted by one to three C₁-C₆ alkyls.

In one or more of the foregoing embodiments, the piperazinyl is optionally substituted by one to three groups selected from C₁-C₆ alkyl.

In one or more of the foregoing embodiments, the piperidinyl is optionally substituted by one group selected from C₁-C₆ alkyl and —NRₐR_{b}, wherein Rₐ and R_{b} are each independently H and C₁-C₆ alkyl.

In one or more of the foregoing embodiments, R₄′ and R₅′ are independently selected from H and optionally substituted C₁-C₆ alkyl. Preferably, R₄′ and R₅′ are H and optionally substituted C₁-C₆ alkyl.

In one or more embodiments, preferred compounds of Formula II include, without limitation:
6-(2-chloro-6-fluorophenyl)-2-((2,4,4-trimethyl-1,2,3,4-tetrahydroisoquinolin-7-yl)amino)imidazo[1,2-b]pyrimido[4,5-d]pyridazin-5(6H)-one;
6-(2,6-difluorophenyl)-2-((3-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3,5-dimethyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((2,4,4-trimethyl-1,2,3,4-tetrahydroisoquinolin-7-yl)amino)imidazo[1,2-b]pyrimido[4,5-d]pyridazin-5(6H)-one;
6-(2,6-difluorophenyl)-2-((3,5-dimethyl-4-(4-methylpiperazin-1-yl)phenyl)amino)imidazo[1,2-b]pyrimido[4,5-d]pyridazin-5(6H)-one;
6-(2-fluoro-6-methylphenyl)-2-((3-methyl-4-(4-methylpiperazin-1-yl)phenyl)amino)imidazo[1,2-b]pyrimido[4,5-d]pyridazin-5(6H)-one;
6-(2-fluoro-6-methylphenyl)-2-((3,5-dimethyl-4-(4-methylpiperazin-1-yl)phenyl)amino) imidazo[1,2-b]pyrimido[4,5-d]pyridazin-5(6H)-one;
6-(2-chloro-6-methylphenyl)-2-((3-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3,5-dimethyl-4-(4-methylpiperazin-1-yl)phenyl)amino)imidazo[1,2-b]pyrimido[4,5-d]pyridazin-5(6H)-one;
6-(2,6-difluorophenyl)-2-((3-chloro-4-(4-methylpiperazin-1-yl) phenyl)amino)imidazo[1,2-b]pyrimido[4,5-d]pyridazin-5(6H)-one;
6-(2,6-difluorophenyl)-2-((3-fluoro-5-methyl-4-(4-methylpiperazin-1-yl)phenyl)amino)imidazo[1,2-b]pyrimido[4,5-d]pyridazin-5(6H)-one;
6-(2,6-difluorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-chloro-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-fluoro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-methyl-4-((3S,5R)-3,4,5-trimethylpiperazin-1-yl)phenyl)amino)imidazo[1,2-b]pyrimido[4,5-d]pyridazin-5(6H)-one;
6-(2-chloro-6-fluorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-methyl-4-(piperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)imidazo[1,2-b]pyrimido[4,5-d]pyridazin-5(6H)-one;
6-(2,6-dichlorophenyl)-2-((3-fluoro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-fluoro-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-chloro-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-methyl-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-methyl-4-((3S,5R)-4-isopropyl-3,5-dimethylpiperazin-1-yl)phenyl)amino)imidazo[1,2-b]pyrimido[4,5-d]pyridazin-5(6H)-one;
6-(2,6-dichlorophenyl)-2-((3-chloro-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-chloro-5-methoxy-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-fluoro-4-(1-methylpiperidin-4-yl)phenyl)amino)imidazo[1,2-b]pyrimido[4,5-d]pyridazin-5(6H)-one;
6-(2,6-dichlorophenyl)-2-((3-chloro-4-(1-methylpiperidin-4-yl)phenyl)amino)imidazo[1,2-b]pyrimido[4,5-d]pyridazin-5(6H)-one;
6-(2,6-dichlorophenyl)-2-((3-methyl-4-(1-methylpiperidin-4-yl)phenyl)amino)imidazo[1,2-b]pyrimido[4,5-d]pyridazin-5(6H)-one;
6-(2,6-dichlorophenyl)-2-((3-fluoro-5-methyl-4-(1-methylpiperidin-4-yl)phenyl)amino)imidazo[1,2-b]pyrimido[4,5-d]pyridazin-5(6H)-one;
6-(2,6-dichlorophenyl)-2-((2,4,4,5-tetramethyl-1,2,3,4-tetrahydroisoquinolin-7-yl)amino)imidazo[1,2-b]pyrimido[4,5-d]pyridazin-5(6H)-one;
6-(2,6-dichlorophenyl)-2-((2,5-dimethyl-1,2,3,4-tetrahydroisoquinolin-7-yl)amino)imidazo[1,2-b]pyrimido[4,5-d]pyridazin-5(6H)-one;
6-(2-bromo-6-fluorophenyl)-2-((3-methyl-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-methyl-4-(4-methylpiperazin-1-yl)phenyl)amino)imidazo[1,2-b]pyrimido[4,5-d]pyridazin-5(6H)-one;
6-(2-bromo-6-chlorophenyl)-2-((3-chloro-4-(4-methylpiperazin-1-yl)phenyl)amino)imidazo[1,2-b]pyrimido[4,5-d]pyridazin-5(6H)-one;
6-(2-bromo-6-chlorophenyl)-2-((3-chloro-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-methyl-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2-fluoro-6-methylphenyl)-2-((3-chloro-4-(4-methylpiperazin-1-yl)phenyl)amino)imidazo[1,2-b]pyrimido[4,5-d]pyridazin-5(6H)-one;
6-(2-fluoro-6-methylphenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3-chloro-4-(4-methylpiperazin-1-yl)phenyl)amino)imidazo[1,2-b]pyrimido[4,5-d]pyridazin-5(6H)-one;
6-(2-chloro-6-methylphenyl)-2-((3-fluoro-5-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)-8-methylimidazo[1,2-b]pyrimido[4,5-d]pyridazin-5(6H)-one;
6-(2,6-dichlorophenyl)-2-((3-chloro-4-(4-methylpiperazin-1-yl)phenyl)amino)-9-methylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-methylimidazo[1,2-b]pyrimido[4,5-d]pyridazin-5(6H)-one;
6-(2,6-dichlorophenyl)-2-((3-fluoro-S-methyl-4-(4-(dimethylamino)piperidin-1-yl)phenyl)amino)-9-methylimidazo[1,2-b]pyrimido[4,5-d]pyridazin-5(6H)-one;
6-(2,6-dichlorophenyl)-2-((3-methyl-4-(4-methylpiperazin-1-yl)phenyl)amino)-9-ethylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-ethylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-isopropylimidazo[1,2-b]pyrimido[4,5-d]pyridazin-5(6H)-one:
6-(2,6-dichlorophenyl)-2-((3-methyl-4-(4-methylpiperazin-1-yl)phenyl)amino)-9-(hydroxymethyl)imidazo[1,2-b]pyrimido[4,5-d]pyridazin-5(6H)-one;
6-allyl-2-((3-methyl-4-(4-(dimethylamino)piperidin-1-yl)phenyl)amino)imidazo[1,2-b]pyrimido[4,5-d]pyridazin-5(6H)-one;
   or a pharmaceutically acceptable salt or prodrug thereof.

In another preferred embodiments, the Wee1 kinase inhibitor suitable for the present disclosure is a compound disclosed in WO2021073491, or a pharmaceutically acceptable salt or prodrug thereof, the entire content of which is incorporated herein by reference. More specifically, the Wee1 kinase inhibitor suitable for the present disclosure is a compound represented by the following Formula III:

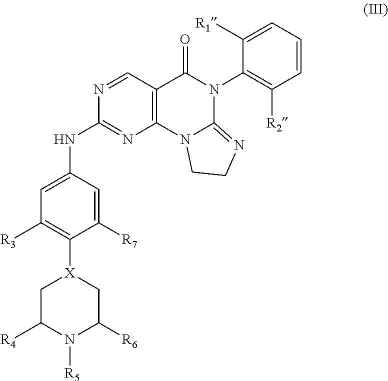

or a stereoisomer thereof, or a pharmaceutically acceptable salt or prodrug thereof, wherein R₁″ and R₂″ are independently halo; R₃″ is halo, C₁₋₄ alkyl or C₁₋4 alkoxy; R₄″ and R₆″ are each independently H or C₁₋₄ alkyl; R₅″ is H or C₁₋₄ alkyl; R₇″ is H, halo, C₁₋₄ alkyl or C₁₋₄ alkoxy; and X is CH or N.

In preferred embodiments of Formula III, R₁″ and R₂″ are both chloro.

In preferred embodiments of Formula III, R₃″ is halo, methyl or ethyl.

In preferred embodiments of Formula III, R₇″ is H, halo, methyl or methoxy.

In preferred embodiments of Formula III, R₄″ and R₆″ are each independently H or methyl.

In preferred embodiments of Formula III, R₅″ is H, methyl or methyl-d3.

In preferred embodiments of Formula III, when X is N, R₄″, R₅″ and R₆″ are not H at the same time; preferably, R₄″ and R₆″ are C₁₋₄ alkyl, and R₅″ is H or C₁₋₄ alkyl; more preferably, R₄″ and R₆″ are methyl, and R₅″ is H, methyl or methyl-d3.

In one or more embodiments, preferred compounds of Formula III include, without limitation:
6-(2,6-dichlorophenyl)-2-((4-((3S,5R)-3,5-dimethylpiperazin-1-yl)-3-methylphenyl)amino)-8,9-dihydroimidazo[1,2-a]pyrimido[5,4-e]pyrimidin-5(6H)-one;
6-(2,6-dichlorophenyl)-2-((4-((3S,5R)-3,5-dimethyl-4-(methyl-d3)piperazin-1-yl)-3-methylphenyl)amino)-8,9-dihydroimidazo[1,2-a]pyrimido[5,4-e]pyrimidin-5(6H)-one;
2-((3-bromo-4-((3S,5R)-3,4,5-trimethylpiperazin-1-yl)phenyl)amino)-6-(2,6-dichlorophenyl)-8,9-dihydroimidazo[1,2-a]pyrimido[5,4-e]pyrimidin-5(6H)-one;
2-((3-bromo-5-methyl-4-((3S,5R)-3,4,5-trimethylpiperazin-1-yl)phenyl)amino)-6-(2,6-dichlorophenyl)-8,9-dihydroimidazo[1,2-a]pyrimido[5,4-e]pyrimidin-5(6H)-one;
6-(2,6-dichlorophenyl)-2-((3-fluoro-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dimethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5S)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(piperidin-4-yl)phenyl)amino)-8,9-dihydroimidazo[1,2-a]pyrimido[5,4-e]pyrimidin-5(6H)-one;
6-(2,6-dichlorophenyl)-2-((3-fluoro-4-(1-methylpiperidin-4-yl)phenyl)amino)-8,9-dihydroimidazo[1,2-a]pyrimido[5,4-e]pyrimidin-5(6H)-one;
2-((3-chloro-4-(1-methylpiperidin-4-yl)phenyl)amino)-6-(2,6-dichlorophenyl)-8,9-dihydroimidazo[1,2-a]pyrimido[5,4-e]pyrimidin-5(6H)-one;
6-(2,6-dichlorophenyl)-2-((3-methyl-4-(1-methylpiperidin-4-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(1-methylpiperidin-4-yl)phenyl)amino)-8,9-dihydroimidazo[1,2-a]pyrimido[5,4-e]pyrimidin-5(6H)-one;
   or a pharmaceutically acceptable salt or prodrug thereof.

Some of the compounds of the present disclosure may exist as stereoisomers including optical isomers The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁₋₄ alcohol according to methods known in the art); esters of hydroxy-containing compounds (e.g., those obtained by condensation with a C₁₋₄ carboxylic acid, C₃₋₆ diacid or anhydride thereof such as succinic anhydride and fumaric anhydride, according to methods known in the art); imines of amino-containing compounds (e.g., those obtained by condensation with a C₁₋₄ aldehyde or ketone according to methods known in the art); carbamate of amino-containing compounds, such as those described by Leu, et al. (J. Med. Chem. 42:3623-3628 (1999)) and Greenwald, et al. (J. Med. Chem. 42:3657-3667 (1999)); and acetals and ketals of alcohol-containing compounds (e.g., those obtained by condensation with chloromethyl methyl ether or chloromethyl ethyl ether according to methods known in the art).

The Wee1 kinase inhibitors of the present disclosure can be prepared using method compounds known to those skilled in the art or methods in the references (including patents, patent applications and patent publications) cited in the present disclosure, including the synthesis methods disclosed in WO2018090939, WO2019011228 and WO2021073491.

The Wee1 kinase inhibitor of the present disclosure can be administered in a pharmaceutical composition containing a pharmaceutically acceptable carrier, wherein the pharmaceutical composition includes all pharmaceutical preparations containing the compound of the present disclosure in an amount that can effectively achieve its intended goal. While individual needs vary, determination of the optimal amount of each part in the pharmaceutical preparation is within the skill of the art. Typically, the compounds or the pharmaceutically acceptable salts thereof may be administered to mammals orally at a dose of about 0.0025 to 50 mg per kg body weight per day. Preferably, from approximately 0.01 mg/kg to approximately 10 mg/kg body weight is orally administered. If a known anticancer agent is also administered, it is administered in an amount that is effective to achieve its intended purpose. The optimal amounts of such known anticancer agents are well known to those skilled in the art.

The unit oral dose may comprise from approximately 0.01 to approximately 50 mg, preferably approximately 0.1 to approximately 10 mg of the compound of the disclosure The unit dose may be administered one or more times, with one or more tablets daily, each containing from approximately 0.1 to 50 mg, conveniently approximately 0.25 to 10 mg of the compound of the disclosure or solvates thereof.

In topical preparations, the compound(s) of the disclosure may be present at a concentration of approximately 0.01 to 100 mg per gram of carrier.

The compound(s) of the disclosure may be administered as a raw chemical. The compounds of the disclosure may also be administered as part of a suitable pharmaceutical preparation containing pharmaceutically acceptable carriers (comprising excipients and auxiliaries). Such pharmaceutically acceptable carriers facilitate the manufacture of pharmaceutically acceptable preparations from the compound(s). Preferably, the pharmaceutical preparations, particularly oral preparations and those used for the preferred administration routes, such as tablets, dragees, and capsules, as well as solutions suitable for injection or oral administration, contain from approximately 0.01% to 99%, preferably from approximately 0.25% to 75% of active compound(s), together with excipient(s).

Also included within the scope of the present disclosure are the pharmaceutically acceptable salts of the Wee1 kinase inhibitor(s) of the present disclosure. Acid addition salts are formed by mixing a solution of the compound(s) of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, hydrocholine, sodium carbonate, tris(hydroxymethyl)aminomethane, N-methyl-glucosamine and the like.

The pharmaceutical preperations of the disclosure may be administered to any mammal, so long as they may experience the therapeutic effects of the compound(s) of the disclosure. Foremost among such mammals are humans and veterinary animals, although the disclosure is not intended to be so limited.

The pharmaceutical preperations of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, frequency of treatment, and the nature of the effect desired.

The pharmaceutical preparations of the present disclosure can be manufactured in a known manner, e.g, by means of conventional mixing, granulating, dragee-making, dissolving, or lyophilizing processes. Pharmaceutical preparations for oral use may be obtained by combining the active compounds with solid excipient(s), optionally grinding the resulting mixture, and processing the mixture of granules, after adding suitable auxiliaries, if desired or necessary, to obtain tablets or dragee cores.

Suitable excipients are, in particular, fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, including maize starch, wheat starch, rice starch, potato starch, gelatin, tragacanth, methylcellulose, hydroxypropylmethylcellulose, sodium carboxymethylcellulose, and/or polyvinyl pyrrolidone. If desired, disintegrating agents may be added, such as the above-mentioned starches and carboxymethyl-starch, cross-linked polyvinyl pyrrolidone, agar, or alginic acid or a salt thereof, such as sodium alginate Auxiliaries are, above all, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose solutions, such as cellulose acetate phthalate or hydroxypropylmethyl-cellulose phthalate, are used. Dyes or pigments may be added to the tablets or dragee core coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate, and stabilizers. In soft capsules, the active compound(s) are preferably dissolved or suspended in suitable liquids, such as fatty oils, or liquid paraffin, in which stabilizers may be added.

Suitable formulations for parenteral administration include aqueous solutions of the active compounds, e.g., aqueous solutions and alkaline solutions of water-soluble salts. In addition, appropriate oily injection suspensions of the active compounds may be administered. Suitable lipophilic solvents or vehicles include fatty oils, e.g., sesame oil, or synthetic fatty acid esters, e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, the suspension may also contain stabilizers.

In accordance with one aspect of the present disclosure, compounds of the disclosure are employed in topical and parenteral formulations and are used for the treatment of skin cancer.

The topical formulations of this disclosure can be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). Preferred carriers are those in which the active ingredient(s) are soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil, such as almond oil, is admixed. A typical example of such a cream is one which includes approximately 40 parts of water, approximately 20 parts of beeswax, approximately 40 parts of mineral oil and approximately one part of almond oil.

Ointments may be formulated by mixing a solution of the active ingredient(s) in a vegetable oil, such as almond oil, with warm soft paraffin and allowing the mixture to cool. A typical example of such an ointment is one which includes approximately 30% by weight of almond oil and approximately 70% by weight of white soft paraffin.

In the present disclosure, “effective amount” refers to an amount of the active compound(s) or pharmaceutical agent(s) that cause the biological or medical response of the tissue, system, animal, or individual human being sought by researchers, veterinarians, physicians, or other clinicians, and the biological or medical response includes one or more of the following: (1) preventing diseases. for example, preventing diseases, conditions, or disorders in an individual who may be susceptible to the disease, condition, or disorder but still not experiencing or presenting the pathology or symptoms of the disease, (2) inhibiting diseases: for example, inhibiting diseases, conditions or disorders in an individual experiencing or presenting the pathology or symptoms of the disease, condition, or disorder (i.e., preventing the further development of the pathology or symptoms of the pathology and/or disorder), and (3) improving diseases: for example, improving diseases, conditions, or disorders in an individual experiencing or presenting the pathology or symptoms of the disease, condition, or disorder (i.e., reverse the pathology and/or symptom). Therefore, a non-limiting example of an “effective dose” of the composition of the present disclosure can be used to inhibit, block or reverse the activation, migration or proliferation of cells, or to effectively treat cancer or improve symptoms of cancer.

The composition containing the Wee1 kinase inhibitor can be used in combination with at least one known anti-cancer drug or a pharmaceutically acceptable salt of an anti-cancer drug. In particular, the composition can be used in combination with other anti-cancer drugs related to the mechanism of DNA damage and repair, including PARP inhibitors olaparib, niraprib, rucaparib, talazoparib, pamiparib, fluzoparib and senaparib, HDAC inhibitors Volinota, Romididesin, Papiseta and Bailesta; and so on. And the composition can be used in combination with other anti-cancer drugs related to cell division checkpoints, including Chk1/2 inhibitors, CDK4/6 inhibitors such as Palbociclib, ATM inhibitors, ATR inhibitors, DNA-PK inhibitors and so on. Other known targeted anti-cancer drugs which may be used for anti-cancer combination therapy include, but are not limited to, PRMT5 inhibitors, Pole inhibitors, RAD51 inhibitors and so on. Other known anti-cancer drugs which may be used for anti-cancer combination therapy include, but are not limited to, alkylating agents, such as busulfan, melphalan, chlorambucil, cyclophosphamide, ifosfamide, temozolomide, bendamustine, cis-platin, mitomycin C, bleomycin and carboplatin; topoisomerase I inhibitors, such as camptothecin, irinotecan and topotecan; topoisomerase II inhibitors, such as doxorubicin, epirubicin, aclacinomycin, mitoxantrone, elliptinium and etoposide; RNA/DNA antimetabolites, such as 5-azacytidine, gemcitabine, 5-fluorouracil and methotrexate; DNA antimetabolites, such as 5-fluoro-2′-deoxy-uridine, fludarabine, nelarabine, ara-C, thioguanine, pralatrexate, pemetrexed, hydroxyurea and thioguanine; antimitotic agent, such as colchicine, vinblastine, vincristine, vinorelbine, paclitaxel, ixabepilone, cabazitaxel and docetaxel; antibodies, such as mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DMI, Ofatumumab, Dinutuximab, Blinatumomab, ipilimumab, avastin, herceptin and mabthera; kinase inhibitors, such as imatinib, gefitinib, erlotinib, osimertinib, afatinib, ceritinib, alectinib, crizotinib, erlotinib, lapatinib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, Idelalisib, pazopanib, Torisel and everolimus. Other known anti-cancer drugs which may be used for anticancer combination therapy include tamoxifen, letrozole, fulvestrant, mitoguazone, octreotide, retinoic acid, arsenic trioxide, zoledronic acid, bortezomib, carfilzomib, Ixazomib, vismodegib, sonidegib, denosumab, thalidomide, lenalidomide, Venetoclax, Aldesleukin (recombinant human interleukin-2) and Sipueucel-T (prostate cancer treatment vaccine).

The present disclosure also provides use of Wee1 kinase inhibitors in the preparation of a medicament for the treatment or prevention of cancer in individuals with H3K27M mutation in the cancer cells. Preferably, the Wee1 kinase inhibitor is a compound of Formula I, II or III, or a stereoisomer, a pharmaceutically acceptable salt or a prodrug thereof as described herein. The present disclosure also provides Wee1 kinase inhibitors or pharmaceutical compositions or pharmaceutical preparations containing one or more Wee1 kinase inhibitors in a method for treating or preventing cancer in individuals with H3K27M mutation in the cancer cells; preferably, the Wee1 kinase inhibitor is a compound of Formula I, Il or III, or a stereoisomer, a pharmaceutically acceptable salt or a prodrug thereof as described herein.

The present disclosure also provides a method of treating or preventing a cancer in an individual with H3K27M mutation in the cancer cells, comprising administering to the individual an effective amount of a Wee1 kinase inhibitor. Preferably, the Wee1 kinase inhibitor is a compound of Formula I, II or III, or a stereoisomer, a pharmaceutically acceptable salt or a prodrug thereof, or a pharmaceutical composition or pharmaceutical preparation thereof as described herein.

The cancer with H3K27M mutation in the cancer cells as described herein may include any type of solid tumors and malignant lymphomas, especially gliomas, including but is not limited to glioblastoma, childhood diffuse pontine glioma and diffuse midline glioma. In preferred embodiments, the cancer with H3K27M mutation is diffuse midline glioma. In some embodiments, the cancers described herein may also include liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, lung cancer, Wilms tumor, cervical cancer, testicular cancer, soft tissue sarcoma, chronic lymphocytic leukemia, primary macroglobulinemia, bladder cancer, chronic myeloid leukemia, primary brain cancer, malignant melanoma, small cell lung cancer, gastric cancer, colon cancer, malignant pancreatic islet tumor, malignant carcinoid cancer, malignant melanoma, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer, as long as it has H3K27M mutation.

Optionally or additionally, in the cancer cells of the cancer described herein, there may also be one or more mutations selected from the group consisting of TP53 mutation, ATRX deletion or mutation, PDGFRA or KIT or KDR amplification, BRCA2 deletion, and SRC amplification. The “mutation”, “deletion” and “amplification” as used herein generally refer to the occurrence of mutations (substitutions and/or insertions), deletions or amplifications known in the art that are associated with the occurrence and development of cancer. “Gene amplification” usually refers to a process in which the copy number of a gene encoding a specific protein is selectively increased while other genes are not increased proportionally.

In some embodiments, oligodendrocyte precursor cells of the individual contain the H3K27M mutation.

In some embodiments, provided are use of a Wee1 kinase inhibitor as described herein in the manufacture of a medicament for treating or preventing pilocytic astrocytoma, germ cell tumor or supratentorial anaplastic ependymoma in a subject in need thereof, a Wee1 kinase inhibitor or its pharmaceutical composition containing the same for use in a method for treating or preventing pilocytic astrocytoma, germ cell tumor or supratentorial anaplastic ependymoma in a subject in need thereof, and a method for treating or preventing pilocytic astrocytoma, germ cell tumor or supratentorial anaplastic ependymoma in a subject in need thereof, comprising administering to the subject an effective amount of a Wee1 kinase inhibitor or its pharmaceutical composition. Preferably, the Wee1 kinase inhibitor is a compound of Formula I, II or III, or a stereoisomer, a pharmaceutically acceptable salt or a prodrug thereof as described herein.

The present disclosure also provides a method for predicting the therapeutic efficacy of a Wee1 kinase inhibitor in a cancer patient, comprising determining whether the patient has H3K27M mutation, wherein the presence of the H3K27M mutation indicates that the cancer patient responds to the Wee1 kinase inhibitor treatment. Methods known in the art can be used to determine whether a patient has H3K27M mutation, and exemplary methods include whole exome sequencing (WES) and the like. Preferably, the Wee1 kinase inhibitor is a compound of Formula I, II or III, or a pharmaceutically acceptable salt or prodrug thereof, or a pharmaceutical composition thereof as described herein. In some embodiments, the oligodendrocyte precursor cells of the patient are detected to determine whether they contain the H3K27M mutation, and presence of the H3K27M mutation in the oligodendrocyte precursor cells indicate response of the patient to the Wee1 kinase inhibitor.

The present disclosure also provides use of a reagent for detecting the presence of H3K27M mutation in individual cancer cells or cancer tissues in the preparation of a detection kit for determining whether an individual responds to or benefits from Wee1 kinase inhibitor treatment. The reagent may be, for example, a reagent for implementing a suitable detection method, and the detection method includes, but is not limited to, whole exon detection. Preferably, the Wee1 kinase inhibitor is a compound of Formula I, II or III, or a pharmaceutically acceptable salt or prodrug thereof, or a pharmaceutical composition thereof as described herein. Exemplary reagents include one or more of the following reagents: reagents for preparing single cell suspension from tissue samples, reagents for extracting nucleic acid from samples, reagents for DNA interruption, terminal repair, addition of A and linker connection, linker sequence, reagents for DNA amplification, probes, reagents for sequencing in sequencer, etc. The sample herein may be a cancer tissue sample. In some embodiments, the sample is a blood sample. In some embodiments, the cell is an oligodendrocyte precursor cell.

The following examples are illustrative, but not limiting, of the methods and preparations of the present disclosure Other suitable modifications and adaptations of various conditions and parameters normally encountered in clinical therapy and which are obvious to those skilled in the art are within the spirit and scope of the disclosure.

### EXAMPLE 1

### Testing the Efficacy of Wee1 Kinase Inhibitor Compound A on Brain Tumor Samples in a MiniPDX™ Drug Sensitivity Model

Mini patient derived xenograft (MiniPDX™) model is a new method to test drug efficacy on human tumors in animal. Individual human tumor samples and patient-derived xenograft (PDX) tumor samples were placed into a small container and transplanted into immunodeficient mice. Using this model, drug effects on the growth and survival of human tumor cells were tested in a mouse animal setting.

A proprietary preparation method was used to isolate tumor cells from fresh human tumor specimens. The cell preparation was placed in a dedicated MiniPDX™ device and transplanted subcutaneously in mice. The specially designed pore size of these MiniPDX™ device allows small molecules below 500 KD to enter and exit freely while tumor cells remain in the device. In the following experiments described below mice were administered with testing drugs systemically for 7 days. After the treatment, the MiniPDX™ devices were taken out, and the tumor cell viability was determined by an in vitro ATP method. The effectiveness of dosage regimens was calculated based on the overall viability of tumor cells.

The efficacy of Wee1 kinase inhibitor 6-(2,6-dichlorophenyl)-2-((3-methyl-4-((3S,5R)-3,4,5-trimethylpiperazin-1-yl)phenyl)amino)-8,9-dihydroimidazo[1,2-a]pyrimidino[5,4-e]pyrimidin-5(6H)-one (Compound A. Example 77 of WO2018090939) on 13 human brain tumors and 4 samples of Diffuse Midline Gliomas (DMG) PDX model was tested using the MiniPDX™ animal model in the present disclosure.

### Experimental Animals:

Female Balb/c-nude mice, 6-8 weeks old weighing 18-22 g, were purchased from GemPharmatech Co., Ltd. Animals were housed for at least 3 days for adaption before experiment.

### Animal Housing and Care:

All the animal studies were carried out following AAALAC guidance. The animals used in the studies were kept in experimental animal center which has passed AAALAC intemational certification. The experimental animal center has an independent ventilation system where the temperature is maintained at 20-26° C., the humidity is maintained at 40-70%, the ventilation is maintained at 15 times/hour, and the day/night cycle is 12 h/12 h. Commercial food and sterilized water were continuously supplied.

### Testing Sample:

Compound A: 6-(2,6-dichlorophenyl)-2-((3-methyl-4-((3S,5R)-3,4,5-trimethylpiperazin-1-yl)phenyl)amino)-8,9-dihydroimidazo[1,2-a]pyrimidino[5,4-e]pyrimidin-5(6H)-one (Example 77 of WO2018090939)

### Vehicle:

0.5% MC (with 0.2% Tween 80) solution.
   Samples of Patient tumor and DMG PDX Model:

TABLE 1
Information of patient tumor sample

| Sample | Cancer | Sample | Patient | Pathological |
|---|---|---|---|---|
| example | type | type | information | results |
| 1 | Brain tumor | Surgical sample | Female, 6 years old | Pilocytic astrocytoma |
| 2 | Brain stem space-occupying | Biopsy sample | Male, 5 years old | Diffuse midline glioma |
| 3 | Brain stem tumor | Biopsy sample | Male, 10 years old | Diffuse midline glioma |
| 4 | Brain germ cell tumor | Surgical sample | Female, 4 years old | Germ cell tumor |
| 5 | Brain stem space-occupying | Biopsy sample | Male, 6 years old | Diffuse midline glioma |
| 6 | Brain stem space-occupying | Biopsy sample | Male, 7 years old | Diffuse midline glioma |
| 7 | Brain germ cell tumor | Surgical sample | Male, 8 years old | Germ cell tumor |
| 8 | Sellar region space- | Surgical sample | Male, 1 year old | Pilocytic mucinous |
| | occupying | | | astrocytoma |
| 9 | Fourth ventricle space- | Surgical sample | Male, 7 years old | Classic |
| | occupying | | | medulloblastoma |
| 10 | Brain stem space-occupying | Biopsy sample | Female, 8 years old | Diffuse midline glioma |
| 11 | Posterior fossa space- | Surgical sample | Male, 4 years old | Pro-fibrosis/nodular |
| | occupying | | | medulloblastoma |
| 12 | Intracalvarium-occupying | Surgical sample | Female, 1 year old | supratentorial anaplastic |
| | | | | ependymoma |
| 13 | Left lateral ventricle - | Surgical sample | Female, 10 years old | subependymal giant cell |
| | occupying | | | astrocytoma |
| 14 | Brain tumor | PDX sample | Female, 5 years old | Diffuse midline glioma |
| 15 | Brain tumor | PDX sample | Male, 9 years old | Diffuse midline glioma |
| 16 | Brain tumor | PDX sample | Male, 2 years old | Diffuse midline glioma |
| 17 | Brain tumor | PDX sample | Female, 4 years old | Diffuse midline glioma |

### Experimental Method:

1. The patient's surgical sample or PDX sample was immersed in HBSS sample buffer.
2. After washing and removing tissue debris, cell suspension was prepared with MiniPDX™ sample preparation buffer and the cells were counted.
3. Certain number of cells were filled into the MiniPDX™ device. The MiniPDX™ device containing human tumor cells was inoculate into mice subcutaneously, and the animals were administered with testing drugs according to the experimental protocol. The inoculation day was designated as the Oth day. The control group was administered with equal volume of vehicle and the treatment group with compound A at 60 mg/kg, P.O., QD for 7 days.
4. At the end of experiment, the MiniPDX™ devices were taken out and cell viability test was performed using a CellTiter-Glo test method.

Drug efficacy was evaluated using the relative increase rate (T/C %) of tumor cells.

The definition of relative increasing rate: T/C %=T/C×100% where T and C are the cell numbers of the treated group and the control group, respectively. The lower the value, the higher the inhibition rate of the drug/combination treatment on tumor growth.

### Results:

Table 2 summarizes the results (T/C %) of Compound A on total 17 brain tumor samples. The results indicate that 9 tumor samples responded to Wee1 kinase inhibitor Compound A with a T/C% less than 50%. Of the 9 tumor samples, 6 tumor samples were DMG tumor with H3K27M mutations.

### EXAMPLE 2

### Whole Exome Sequencing (WES)

WES was performed on the 15 brain tumor samples.

### The Experimental Method:

Fresh human tumor samples were prepared into cell suspension. About 2000-5000 cells were used for nucleic acid extraction. The extracted DNA was amplified by breaking, end repairing, adding A, and connecting with adapters to prepare a library, and then captured and purified into the final sequencing library by exome probe. After identification, it was sequenced on the Illumina sequencer and off-machine data analysis was performed.

The alterations of 140 tumor driver genes of each sample are analyzed. The altered genes detected in 3 or more samples are shown in Table 2 below. We performed both t-test and Mann Whitney Wilcoxon test to examine the significance of correlation between altered gene and compound inhibitory effect.

TABLE 2
Test Results

| | | | | | ATRX | PDGFRA/ | | | | |
|---|---|---|---|---|---|---|---|---|---|---|
| Sample | Pathological | | H3F3A | TP53 | deletion/ | KIT/KDR | BRCA2 | SRC | KDM6A | FAT1 |
| example | results | T/C % | mutation | mutation | mutation | amplification | deletion | amplification | deletion | mutation |
| 1 | Pilocytic | 11.20% | / | / | / | / | / | / | / | / |
| | astrocytoma | | | | | | | | | |
| 2 | Diffuse | 19.70% | K27M | Y | / | Y | Y | Y | / | / |
| | midline | | | | | | | | | |
| | glioma | | | | | | | | | |
| 3 | Diffuse | 26.11% | K27M | Y | / | / | / | Y | / | / |
| | midline | | | | | | | | | |
| | glioma | | | | | | | | | |
| 4 | Germ cell | 31.15% | / | / | / | / | / | / | / | / |
| | tumor | | | | | | | | | |
| 5 | Diffuse | 38.66% | K27M | Y | ATRX | / | / | Y | / | / |
| | midline | | | | mutation | | | | | |
| | glioma | | | | | | | | | |
| 6 | Diffuse | 47.16% | K27M | Y | / | / | / | / | / | / |
| | midline | | | | | | | | | |
| | glioma | | | | | | | | | |
| 7 | Germ cell | 85.55% | / | / | / | Y | Y | / | Y | / |
| | tumor | | | | | | | | | |
| 8 | Pilocytic | 102.24% | / | / | ATRX | / | / | / | Y | / |
| | mucinous | | | | deletion | | | | | |
| | astrocytoma | | | | | | | | | |
| 9 | Classic | 116.23% | / | / | ATRX | / | / | / | Y | / |
| | medulloblastoma | | | | deletion | | | | | |
| 10 | Diffuse | 129.97% | / | Y | / | / | Y | / | / | Y |
| | midline | | | | | | | | | |
| | glioma | | | | | | | | | |
| 11 | Pro- | 142.47% | / | Y | / | Y | / | / | / | Y |
| | fibrosis/ | | | | | | | | | |
| | nodular | | | | | | | | | |
| | medulloblastoma | | | | | | | | | |
| 12 | supratentorial | 26.48% | / | N/A | N/A | N/A | N/A | N/A | N/A | N/A |
| | anaplastic | | | | | | | | | |
| | ependymoma | | | | | | | | | |
| 13 | subependymal | 126.32% | / | N/A | N/A | N/A | N/A | N/A | N/A | N/A |
| | giant cell | | | | | | | | | |
| | astrocytoma | | | | | | | | | |
| 14 | Diffuse | 40% | K27M | Y | / | / | / | / | / | / |
| | midline | | | | | | | | | |
| | glioma | | | | | | | | | |
| 15 | Diffuse | 0 | K27M | Y | / | / | / | / | / | / |
| | midline | | | | | | | | | |
| | glioma | | | | | | | | | |
| 16 | High | 62% | K27M | Y | / | / | / | / | / | / |
| | grade | | | | | | | | | |
| | spinal | | | | | | | | | |
| | tumor | | | | | | | | | |
| 17 | Embryonal | 84% | K27M | Y | / | / | / | / | / | / |
| | tumor | | | | | | | | | |

### Results:

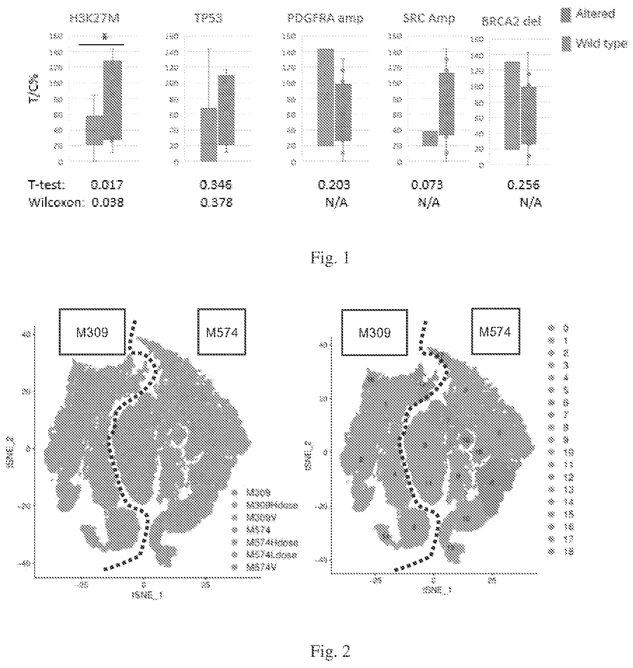

Due to low quality of seq data, two samples (sample 12 and 13) were excluded from correlation analyses for TP53 and PDGFRA. The wild-type status of H3F3A of these two samples are from the results of clinical test. Among 140 tumor driver genes, 8 genes were found altered in 3 or more samples. Both T-test and Mann-Whiteney Wilcoxon Rank test were carried out to examine the significance of the correlation of a specific altered gene in drug response (Mann-Whiteney Wilcoxon Rank test was carried out to examine only H3K27M and TP53 because there are not sufficient amounts of samples for PDGFRA amplification, SRC amplification and BRCA2 deletion). Among tested genes, K27M is the only altered gene that significantly correlated with the compound inhibitory effect in both tests (t-test: 0.017, Wilcoxon: 0.038) (FIG. 1). The correlation of compound inhibition with TP53 mutation, PDGFRA amplification, SRC amplification or BRCA2 deletion is not significant (FIG. 1).

In summary, we have found that Wee1 kinase inhibitor Compound A had a significant inhibitory effect on human DMG tumor cells with H3K27M mutation. The H3K27M mutation may be used as an effective screening biomarker of DMG patients in the clinic when Wee1 kinase inhibitors are used.

### EXAMPLE 3

### The In Vivo Efficacy of Wee1 Kinase Inhibitor Compound A in Two Human Brain Tumor PDX Models With Different H3K27M Ratio

The in vivo efficacy of Wee1 kinase inhibitor Compound A on human brain tumors was conducted on 2 DMG patient-derived xenograft (PDX) mouse models with various level of H3K27M in terms of tumor growth inhibition (TGI).

### Experimental Animals:

Female Nu/Nu mice, 5-8 weeks old weighing 19-25 g, were purchased from Zhejiang Vital-River Co., Ltd. Animals were housed for at least 3 days for adaption before experiment.

### Animal Housing and Care:

All the animal studies were carried out following AAALAC guidance. The animals used in the studies were kept in experimental animal center which has passed AAALAC international certification. The experimental animal center has an independent ventilation system where the temperature is maintained at 20-26° C., the humidity is maintained at 40-70%, the ventilation is maintained at 15 times/hour, and the day/night cycle is 12 h/12 h. Commercial food and sterilized water were continuously supplied.

### Testing Sample:

Compound A: 6-(2,6-dichlorophenyl)-2-((3-methyl-4-((3S,5R)-3,4,5-trimethylpiperazin-1-yl)phenyl)amino)-8,9-dihydroimidazo[1,2-a]pyrimidino[5,4-e]pyrimidin-5(6H)-one (Example 77 of WO2018090939)

### Vehicle:

0.5% MC solution.

### PDX Model Sample:

TABLE 3
Information of PDX model

| | Patient | Patient | H3K27M mut. Frequency % | |
|---|---|---|---|---|
| PDX Model ID | age | Gender | H3F3A isoform | H3C3 isoform |
| M309 | 5 | Female | None | 20.4 |
| M574 | 2 | Male | 79.5 | None |

### Experiments and Results:

In M309 PDX model, tumor-bearing mice were treated with vehicle control or 100 mg/kg Compound A (n=3/group) at a dosing schedule of QD for 3 days on, 4 days off for four weeks. A TGI value of 38.71% was achieved and the treatment appears tolerable overall based on the associated percent body weight change (loss).

In M574 PDX model, tumor-bearing mice were orally administered 100 mg/kg Compound A (n=8/group) at a dosing schedule of QD for 3 days on, 4 days off for four weeks exhibited a reduction in percent body weight compared to vehicle-treated animals. Note that the decrease in average body weight of the 100 mg/kg-treated animals did not exceed 5%. The results also show the significant (p<0.01) reduction in tumor volume relative to control, corresponding to 58.0% tumor growth inhibition (TGI) following 100 mg/kg Compound A treatment.

In summary, the data of PDX models strongly indicate that Wee1 kinase inhibitor (Compound A) had a significant inhibitory effect on human tumor cells, which is correlated with the high percentage of H3K27M mutation of tumor sample.

### EXAMPLE 4

### scRNA seq

The cellular mechanism underlying the response of brain tumor cells with H3K27M to Compound A was investigated via scRNA seq. Two Key clinical features of Diffuse Midline Gliomas (DMG) are the restricted development window and their specific midline location, which implicate a developmentally early specific cell of origin. Two types of brain precursor cells, neuronal precursor cells (NPC) and oligodendrocyte precursor like cells (OPC-like), are demonstrated as the tumor origin of H3K27M-gliomas (Kosuke, Science 2014; Filbin, Science 2018). To investigate what type of the brain precursor cells with H3K27M responds to compound and what mechanism underlying the sensitivity to compound, seRNA-seq analyses were carried out to characterize each specific cell type from a heterogenous populations of 2 DMG PDX model in pre- and post-compound treatment.

### Methods:

### Tissue Dissociation and Preparation of Single-Cell Suspension

Three samples (pretreatment, 28 days of post-treatment vehicle and post-treatment compound) separately from two DMG PDX models (M309 and M574) were used The subcutaneous xenograft samples were dissected from mouse and then transferred into a sterile RNase-free culture dish containing an appropriate amount of calcium-free and magnesium-free 1×PBS on ice. After removing non-purpose tissues (blood stains and fatty layers), the tumor tissues were cut into 0.5 mm² pieces and washed with 1×PBS for dissociation into single cells. Tissues were dissociated into single cells in dissociation solution (0.35% collagenase IV5, 2 mg/mL papain, 120 Units/mL DNase I) in 37° C.water bath with shaking for 20 min at 100 rpm. Digestion was terminated with 1×PBS containing 10% fetal bovine serum (FBS, V/V), then pipetting 5-10 times with a Pasteur pipette. The resulting cell suspension was filtered by passing through 70-30 μm stacked cell strainer and centrifuged at 300 g for 5 min at 4° C. The cell pellet was resuspended in 100 μL 1×PBS (0.04% BSA) and added with 1 mL 1×red blood cell lysis buffer (MACS 130-094-183, 10×) and incubated at room temperature or on ice for 2-10 min to lyse remaining red blood cells. After incubation, the suspension was centrifuged at 300 g for 5 min at room temperature. The suspension was resuspended in 100 μL Dead Cell Removal MicroBeads (MACS 130-090-101) and remove dead cells using Miltenyi® Dead Cell Removal Kit (MACS 130-090-101). Then the suspension was resuspended in 1×PBS (0.04% BSA) and centrifuged at 300 g for 3 min at 4° C. (repeat twice). The cell pellet was resuspended in 50 μL of 1×PBS (0.04% BSA). The overall cell viability was confirmed by trypan blue exclusion, which needed to be above 85%, single cell suspensions were counted using a haemocytometer/Countess II Automated Cell Counter and concentration adjusted to 700-1200 cells/μL.

### Chromium 10× Genomics Library and Sequencing

Single-cell suspensions were loaded to 10× Chromium to capture 4500-9000 single cell according to the manufacturer's instructions of 10× Genomics Chromium Single-Cell 3′kit (V3). cDNA amplification and library construction steps were performed according to the standard protocol. Sequencing was performed on an Illumina NovaSeq 6000 sequencing system (paired-end multiplexing run, 150 bp) by LC-BioTechnology Co. Ltd., (Hangzhou, China) at a minimum depth of 20,000 reads per cell.

scRNA-seq Data Processing and Analysis

The XenoCell (v 1.0) is used to differ the origin of reads. The cellranger (v 6.0.2) package was used to process raw extract human-origin reads to a cell-transcript count matrix. Scater (v.1.18 6) was finally used to screen cells Alignment was performed to the hg38 annotation. Viable single cells were identified based on total mRNA abundance, number of unique transcripts, and mitochondria fraction. Markov affinity-based Graph Imputation of Cells was used to overcome the missing transcripts, typically occurred in scRNA-seq, and to de-noise the data.

All cells were visualized using t-Distributed Stochastic Neighbor Embedding (t-SNE) computed on the first 30 principal components of the imputed count matrix. To identify three types of brain progenitor cells (astrocyte cell AC, oligodendrocyte precursor cell OPC, and neuronal precursor cell NPC), gene sets identified in the studies of dissected human developing brains were used in this analysis. Cell-cycle gene sets obtained from Genomes pathway and gene ontology analysis were used to identify cell cycle cells Gene sets identified in the studies of dissected human DMG cells were used to identify cells with H3K27M.

### Results

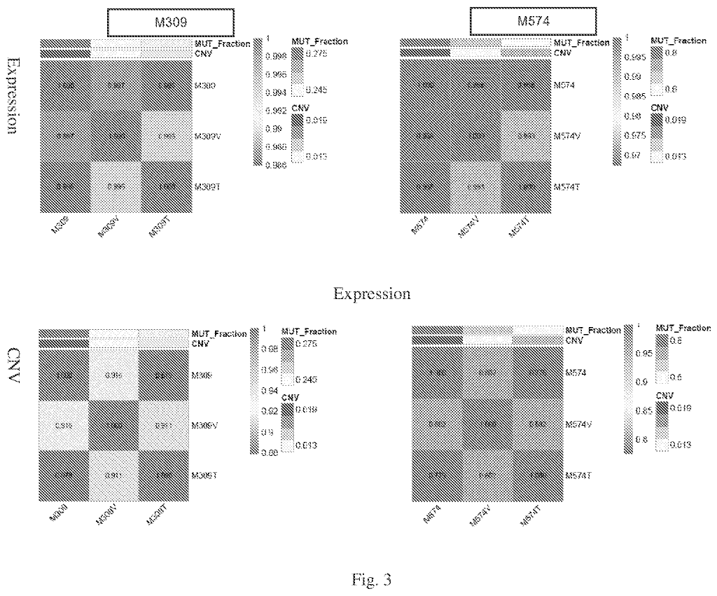

scRNA-seq were carried out on total 6 samples of 2 human brain PDX models (M309 and M574 profiled in Table 3, profile of cell number of each sample is listed in Table 4). A total of 44927 viable cells that passed quality control were used in the analysis. Samples are analyzed based on the model-origin due to rare overlapping between two models in the combined analyses (FIG. 2). Cells containing of H3K27M were inferred by the distribution of markers genes with 0.99 as a cutoff value. The ratio of H3K27M in pretreatment sample of M309 and M574 is 28% and 80%, respectively, which are close to the ratio of H3K27M in the primary tumor sample. The comparison of gene expression of inferred cells containing H3K27M or wild type with one of matched primary tumor sample was carried out. The strong correlation is found. These data strongly validate the approach that was used to identify cell with H3K27M in this scRNA-seq analyses (FIG. 3).

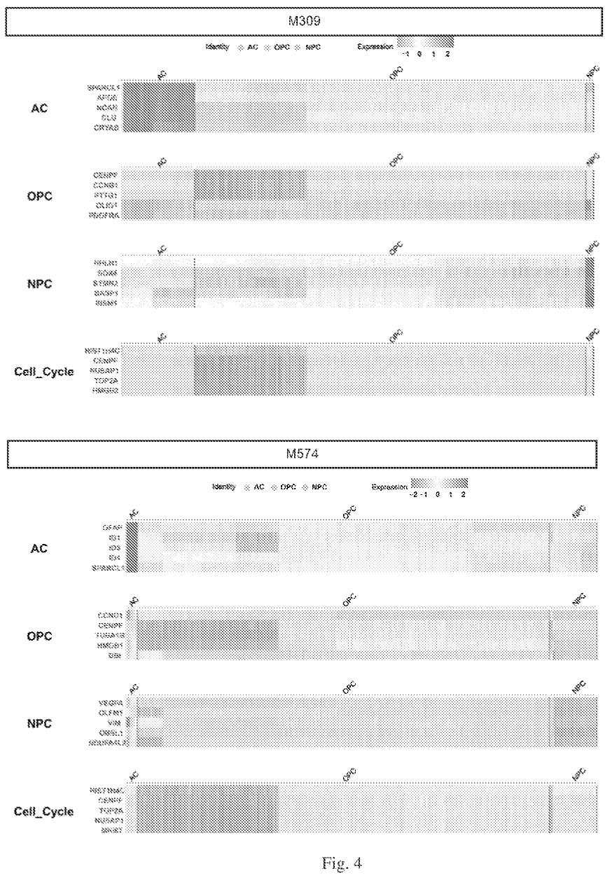

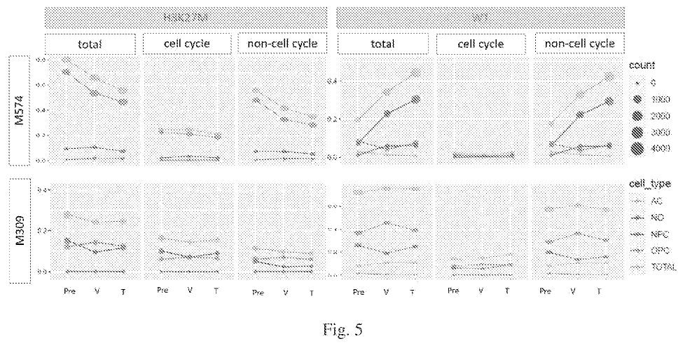

Patterns of heterogeneity were then examined and three type of brain precursor cells were consistently observed in all of samples The malignant cells with or without H3K27M were further analyzed based on the cell type and the status of cell cycle (FIG. 4). In both MS74 and M309, OPC cells with H3K27M are the major cell type contributing to the H3K27M-containing cell number drop (FIG. 5). The results of scRNA-seq analyses demonstrate that OPC with H3K27M are the major cell type responding to the compound inhibition.

TABLE 4
Number of cells from each sample used in scRNA-seq analyses

| Sample | raw | qc.lib | qc.nexprs | qe.mito | AILPASS |
|---|---|---|---|---|---|
| M309 | 9856 | 9304 | 9260 | 9272 | 9008 |
| M309V | 4814 | 4469 | 4454 | 4609 | 4331 |
| M309T | 5357 | 4954 | 4934 | 5153 | 4858 |
| M574 | 4549 | 4299 | 4168 | 4287 | 4003 |
| M574V | 7471 | 6867 | 6898 | 7129 | 6783 |
| M574T | 9502 | 8671 | 8862 | 8889 | 8438 |

V: vehicle; T: treatment.

The sc-RNA seq results strongly support that H3K27M may use as a predicting biomarker for the treatment of DMG with the compound.

WES was performed on the 2 xenografted tumor samples (M574).

### The Experimental Method:

Xenografted tumor samples (1 vehicle and 1 treatment sample) were separately stored in RNAlater and all were used for DNA extraction. 300 ng of the extracted DNA was amplified by breaking, end repairing, and connecting with adapters to prepare a library, and then captured and purified into the final sequencing library by exome probe. After identification, it was sequenced on the Illumina sequencer and off-machine data analysis was performed.

The frequency of H3K27M in each sample is calculated by the ratio of the number of sequence reads with mutant to total number of sequence reads that map to the location of H3K27.

### Results:

The frequency of H3K27M in vehicle and treatment sample was 71% and 54.5% respectively. The frequency of H3K27M dropped after compound treatment.

1-8. (canceled)

9. A method for treating or preventing a cancer in an individual with H3K27M mutation in the cancer cells or with pilocytic astrocytoma, germ cell tumor or supratentorial anaplastic ependymoma, comprising administering to the individual an effective amount of a Wee1 kinase inhibitor or a pharmaceutical composition comprising the effective amount of the Wee1 kinase inhibitor.

10-11. (canceled)

12. The method of claim 9, wherein:
the individual is further treated with radiotherapy; and/or
the individual is further administered with at least one known anti-cancer drug, or a pharmaceutically acceptable salt of the anti-cancer drug.

13. A method for predicting the therapeutic efficacy of a Wee1 kinase inhibitor in a cancer patient, comprising determining whether the patient has a H3K27M mutation; wherein the presence of the H3K27M mutation indicates that the patient responds to the Wee1 kinase inhibitor treatment.

14-15. (canceled)

16. The method of claim 9, wherein the Wee1 kinase inhibitor is selected from compounds of Formula I: 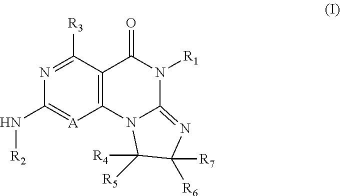
or pharmaceutically acceptable salts or prodrugs thereof, wherein:
A is N or CR₁₅;
R₁ is H, optionally substituted C₁₋₈ alkyl, optionally substituted C₂₋₈ alkenyl, optionally substituted C₃₋₈ cycloalkyl, optionally substituted aryl, or optionally substituted heteroaryl;
R₂ is an optionally substituted heterocyclic group, an optionally substituted aryl, or an optionally substituted heteroaryl;
R₃-R₇ and R₁₅ are independently H, halo, optionally substituted amino, optionally substituted alkoxy, optionally substituted C₁₋₁₀ alkyl, haloalkyl, alkenyl, alkynyl, hydroxylalkyl, aminoalkyl, carboxylalkyl, nitro, cyano, acylamido, hydroxy, thiol, acyloxy, azido, carboxy, ethylenedioxo, carbonylamido or optionally substituted alkylthiol;
or the Wee1 kinase inhibitor is selected from compounds of Formula II: 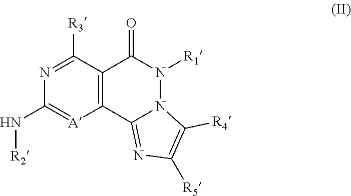
or pharmaceutically acceptable salts or prodrugs thereof, wherein:
A′ is N or CR₆′;
R₁′is H, optionally substituted C₁-C₈ alkyl, optionally substituted C₂-C₈ alkenyl, optionally substituted C₃-C₈ cycloalkyl, optionally substituted aryl, optionally substituted heterocyclic group or optionally substituted heteroaryl;
R₂′ is an optionally substituted carbocyclic group, an optionally substituted heterocyclic group, an optionally substituted aryl, or an optionally substituted heteroaryl;
R₃′-R₆′ are independently H, halo, optionally substituted amino, optionally substituted alkoxy, optionally substituted C₁-C₁₀ alkyl, alkenyl, alkynyl, nitro, cyano, acylamido, hydroxy, thiol, acyloxy, azido, carboxy, ethylenedioxo, carbonylamido or optionally substituted alkylthiol.

17. The method of claim 16, wherein the optionally substituted C₁-C₁₀ alkyl defined in R₃′-R₆′ is haloalkyl, hydroxylalkyl, aminoalkyl or carboxylalkyl.

18. The method of claim 9, wherein the Wee1 kinase inhibitor is a compound represented by Formula III: 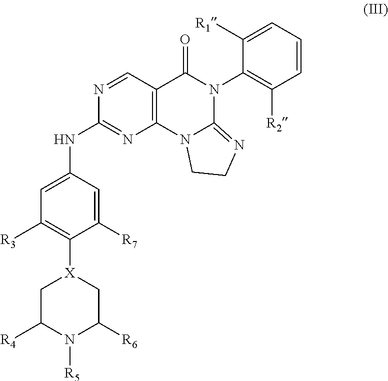
or a stereoisomer thereof, or a pharmaceutically acceptable salt or prodrug thereof, wherein R₁″ and R₂″ are independently halo; R₃″ is halo, C₁₋₄ alkyl or C₁₋₄ alkoxy; R₄″ and R₆″ are each independently H or C₁₋₄ alkyl; R₅″ is H or C₁-4 alkyl; R₇″ is H, halo, C₁₋₄ alkyl or C₁₋₄ alkoxy; and X is CH or N;

19. The method of claim 9, wherein the Wee1 kinase inhibitor is selected from the group consisting of:
6-(2-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-8,9-dihydroimidazo[1,2-a]pyrimido[5,4-e]pyrimidin-5(6H)-one;
4-(2-chlorophenyl)-8-((4-(4-methylpiperazin-1-yl)phenyl)amino)-1,2-dihydroimidazo[1,2-a]pyrido[3,4-e]pyrimidin-5(4H)-one;
6-(2,6-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-isopropylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-acetylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2-chloro-6-fluorophenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2-chloro-6-fluorophenyl)-2-((2′-methyl-2′,3′-dihydro-1′H-spiro[cyclopropane-1,4′-isoquinolin]7′-yl)amino)-8,9-dihydroimidazo[1,2-a]pyrimido[5,4-e]pyrimidin-5(6H)-one;
6-(2-chloro-6-fluorophenyl)-2-((2′-acetyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dimethylphenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dimethylphenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dimethylphenyl)-2-((2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-isopropyl-2-((4-(4-methylpiperazin-1-yl)phenyl)amino)-8,9-dihydroimidazo[1,2-a]pyrimido[5,4-e]pyrimidin-5(6H)-one;
6-tert-butyl-2-((4-(4-methylpiperazin-1-yl)phenyl)amino)-8,9-dihydroimidazo[1,2-a]pyrimido[5,4-e]pyrimidin-5(6H)-one;
6-cyclopropyl-2-((4-(4-methylpiperazin-1-yl)phenyl)amino)-8,9-dihydroimidazo[1,2-a]pyrimido[5,4-e]pyrimidin-5(6H)-one;
6-cyclohexyl-2-((4-(4-methylpiperazin-1-yl)phenyl)amino)-8,9-dihydroimidazo[1,2-a]pyrimido[5,4-e]pyrimidin-5(6H)-one;
6-allyl-2-((4-(4-methylpiperazin-1-yl)phenyl)amino)-8,9-dihydroimidazo[1,2-a]pyrimido[5,4-e]pyrimidin-5(6H)-one;
6-(thiophen-2-yl)-2-((4-(4-methylpiperazin-1-yl)phenyl)amino)-8,9-dihydroimidazo[1,2-a]pyrimido[5,4-e]pyrimidin-5(6H)-one;
6-(furan-2-yl)-2-((4-(4-methylpiperazin-1-yl)phenyl)amino)-8,9-dihydroimidazo[1,2-a]pyrimido[5,4-e]pyrimidin-5(6H)-one;
6-(1H-pyrrol-2-yl)-2-((4-(4-methylpiperazin-1-yl)phenyl)amino)-8,9-dihydroimidazo[1,2-a]pyrimido[5,4-e]pyrimidin-5(6H)-one;
6-(1H-imidazol-5-yl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-8,8-dimethyl-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-9,9-dimethyl-8,9-dihydroimidazo[1,2-a]pyrimido[5,4-e]pyrimidin-5(6H)-one;
6-(2-chloro-6-fluorophenyl)-2-((4-((2S,6R)-2,6-dimethylmorpholino)phenyl)amino)-8,9-dihydroimidazo[1,2-a]pyrimido[5,4-e]pyrimidin-5(6H)-one;
6-(2-chloro-6-fluorophenyl)-2-((4-(morpholinomethyl)phenyl)amino)-8,9-dihydroimidazo[1,2-a]pyrimido[5,4-e]pyrimidin-5(6H)-one;
6-(2-chloro-6-fluorophenyl)-2-((4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-fluoro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-fluoro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-chloro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-2-(trifluoromethyl)phenyl)amino)-8,9-dihydroimidazo[1,2-a]pyrimido[5,4-e]pyrimidin-5(6H)-one;
6-(2-chloro-6-fluorophenyl)-2-((2-trifluoromethyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-3-(trifluoromethyl)phenyl)amino)-8,9-dihydroimidazo[1,2-a]pyrimido[5,4-e]pyrimidin-5(6H)-one;
6-(2-chloro-6-fluorophenyl)-2-((3-trifluoromethyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-isopropylpiperazin-1-yl)-3-methylphenyl)amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)-3-methylphenyl)amino)-8,9-dihydroimidazo[1,2-a]pyrimido[5,4-e]pyrimidin-5(6H)-one;
6-(2-chloro-6-fluorophenyl)-2-((3-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-3-nitrophenyl)amino)-8,9-dihydroimidazo[1,2-a]pyrimido[5,4-e]pyrimidin-5(6H)-one;
6-(2-chloro-6-fluorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((6-(4-methylpiperazin-1-yl)pyridin-3-yl)amino)-8,9-dihydroimidazo[1,2-a]pyrimido[5,4-e]pyrimidin-5(6H)-one;
6-(2-chloro-6-fluorophenyl)-2-((2′-isopropyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2-chloro-6-fluorophenyl)-2-((5-methyl-4,5,6,7-tetrahydropyrazolo[1,5-a]pyrazin-2-yl)amino)-8,9-dihydroimidazo[1,2-a]pyrimido[5,4-e]pyrimidin-5(6H)-one;
6-(2,6-difluorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-(trifluoromethyl)phenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-isopropylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-((3S,5R)-4-isopropyl-3,5-dimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-chloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-trifluoromethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-methylpiperazin-1-yl)-3-(trifluoromethyl)phenyl)amino)-8,9-dihydroimidazo[1,2-a]pyrimido[5,4-e]pyrimidin-5(6H)-one;
6-(2,6-dichlorophenyl)-2-((3-trifluoromethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-isopropylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5R)-4-isopropyl-3,5-dimethylpiperazin-1-yl)-phenyl)amino)-8,9-dihydroimidazo[1,2-a]pyrimido[5,4-e]pyrimidin-5(6H)-one;
6-(2,6-dichlorophenyl)-2-((5-methyl-4,5,6,7-tetrahydropyrazolo[1,5-a]pyrazin-2-yl)amino)-8,9-dihydroimidazo[1,2-a]pyrimido[5,4-e]pyrimidin-5(6H)-one;
6-(2-chloro-6-trifluoromethylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methoxyphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dimethylphenyl)-2-((4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(4-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(3-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(2,4-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-4-fluorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-3-fluorophenyl)-2-((4-(4-methylpiperazin-1-yl)phenyl)amino)-8,9-dihydroimidazo[1,2-a]pyrimido[5,4-e]pyrimidin-5(6H)-one;
6-(2,5-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2,3-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(pyrimidin-2-yl)-2-((4-(4-methylpiperazin-1-yl)phenyl)amino)-8,9-dihydroimidazo[1,2-a]pyrimido[5,4-e]pyrimidin-5(6H)-one;
2-((4-(4-methylpiperazin-1-yl)phenyl)amino)-8,9-dihydroimidazo[1,2-a]pyrimido[5,4-e]pyrimidin-5(6H)-one;
6-cyclobutyl-2-((4-(4-methylpiperazin-1-yl)phenyl)amino)-8,9-dihydroimidazo[1,2-a]pyrimido[5,4-e]pyrimidin-5(6H)-one;
6-cyclopentyl-2-((4-(4-methylpiperazin-1-yl)phenyl)amino)-8,9-dihydroimidazo[1,2-a]pyrimido[5,4-e]pyrimidin-5(6H)-one;
6-phenyl-2-((4-(4-methylpiperazin-1-yl)phenyl)amino)-8,9-dihydroimidazo[1,2-a]pyrimido[5,4-e]pyrimidin-5(6H)-one;
6-(pyridin-2-yl)-2-((4-(4-methylpiperazin-1-yl)phenyl)amino)-8,9-dihydroimidazo[1,2-a]pyrimido[5,4-e]pyrimidin-5(6H)-one;
6-(pyridin-3-yl)-2-((4-(4-methylpiperazin-1-yl)phenyl)amino)-8,9-dihydroimidazo[1,2-a]pyrimido[5,4-e]pyrimidin-5(6H)-one;
6-(pyridin-4-yl)-2-((4-(4-methylpiperazin-1-yl)phenyl)amino)-8,9-dihydroimidazo[1,2-a]pyrimido[5,4-e]pyrimidin-5(6H)-one;
6-(2,6-difluorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2,6-difluorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(1H-imidazol-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methyl-1,4-diazecyclohept-1-yl)phenyl)amino)-8,9-dihydroimidazo[1,2-a]pyrimido[5,4-e]pyrimidin-5(6H)-one;
6-(2-chloro-6-fluorophenyl)-2-((4-((4-methylpiperazin-1-yl)methyl)phenyl)amino)-8,9-dihydroimidazo[1,2-a]pyrimido[5,4-e]pyrimidin-5(6H)-one;
6-(2-chloro-6-fluorophenyl)-2-((4-(2-(dimethylamino)ethoxy)phenyl)amino)-8,9-dihydroimidazo[1,2-a]pyrimido[5,4-e]pyrimidin-5(6H)-one;
6-(2-chloro-6-fluorophenyl)-2-((4-(3-(dimethylamino)propoxy)phenyl)amino)-8,9-dihydroimidazo[1,2-a]pyrimido[5,4-e]pyrimidin-5(6H)-one;
6-(2-chloro-6-fluorophenyl)-2-((4-((1-methylpiperidin-4-yl)oxy)phenyl)amino)-8,9-dihydroimidazo[1,2-a]pyrimido[5,4-e]pyrimidin-5(6H)-one;
6-(2-chloro-6-fluorophenyl)-2-((4-((2-(dimethylamino)ethyl)amino)phenyl)amino)-8,9-dihydroimidazo[1,2-a]pyrimido[5,4-e]pyrimidin-5(6H)-one;
6-(2-chloro-6-fluorophenyl)-2-((4-((2-(dimethylamino)ethyl)(methyl)amino)phenyl)amino)-8,9-dihydroimidazo[1,2-a]pyrimido[5,4-e]pyrimidin-5(6H)-one;
6-(2-chloro-6-fluorophenyl)-2-((4-((3-(dimethylamino)propyl)amino)phenyl)amino)-8,9-dihydroimidazo[1,2-a]pyrimido[5,4-e]pyrimidin-5(6H)-one;
6-(2-chloro-6-fluorophenyl)-2-((4-((3-(dimethylamino)propyl)(methyl)amino)phenyl)amino)-8,9-dihydroimidazo[1,2-a]pyrimido[5,4-e]pyrimidin-5(6H)-one;
6-(2-chloro-6-fluorophenyl)-2-((4-((1-methylpiperidin-4-yl)amino)phenyl)amino)-8,9-dihydroimidazo[1,2-a]pyrimido[5,4-e]pyrimidin-5(6H)-one;
6-(2-chloro-6-fluorophenyl)-2-((4-(methyl(1-methylpiperidin-4-yl)amino)phenyl)amino)-8,9-dihydroimidazo[1,2-a]pyrimido[5,4-e]pyrimidin-5(6H)-one;
6-(2-chloro-6-fluorophenyl)-2-((4-(1-methylpiperidin-4-yl)phenyl)amino)-8,9-dihydroimidazo[1,2-a]pyrimido[5,4-e]pyrimidin-5(6H)-one;
6-(2-chloro-6-fluorophenyl)-2-((4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-bromo-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-(4-(dimethylamino)piperidin-1-yl)-phenyl)amino)-8,9-dihydroimidazo[1,2-a]pyrimido[5,4-e]pyrimidin-5(6H)-one;
6-(2-chloro-6-fluorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((5-(4-methylpiperazin-1-yl)pyrazin-2-yl)amino)-8,9-dihydroimidazo[1,2-a]pyrimido[5,4-e]pyrimidin-5(6H)-one;
6-(2-chloro-6-fluorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-bromo-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-5-trifluoromethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methoxy-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((5-chloro-2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,5-dimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((5-chloro-2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,4,4,5-tetramethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((5′-chloro-2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((2′,5′-dimethyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((3,5-dichloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-bromo-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3,5-dichloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-(dimethylamino)piperidin-1-yl)-5-methoxyphenyl)amino)-8,9-dihydroimidazo[1,2-a]pyrimido[5,4-e]pyrimidin-5(6H)-one;
6-(2,6-dichlorophenyl)-2-((3-bromo-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one; 6-(2,6-dichlorophenyl)-2-((3-fluoro-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-trifluoromethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-chloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-bromo-6-chlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-bromo-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-bromo-5-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-methylphenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-4-(piperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-(hydroxymethyl)-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-(hydroxymethyl)-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-(hydroxymethyl)-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-(2-hydroxyethyl)piperazin-1-yl)phenyl)amino)-8,9-dihydroimidazo[1,2-a]pyrimido[5,4-e]pyrimidin-5(6H)-one;
6-(2,6-dichlorophenyl)-2-((4-morpholinophenyl)amino)-8,9-dihydroimidazo[1,2-a]pyrimido[5,4-e]pyrimidin-5(6H)-one;
6-(2,6-dichlorophenyl)-2-((3-((methylamino)methyl)-4-morpholinophenyl)amino)-8,9-dihydroimidazo[1,2-a]pyrimido[5,4-e]pyrimidin-5(6H)-one;
6-(2-bromo-6-chlorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2,4,4-trimethyl-1,2,3,4-tetrahydroisoquinolin-7-yl)amino)imidazo[1,2-b]pyrimido[4,5-d]pyridazin-5(6H)-one;
6-(2,6-difluorophenyl)-2-((3-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3,5-dimethyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((2,4,4-trimethyl-1,2,3,4-tetrahydroisoquinolin-7-yl)amino)imidazo[1,2-b]pyrimido[4,5-d]pyridazin-5(6H)-one;
6-(2,6-difluorophenyl)-2-((3,5-dimethyl-4-(4-methylpiperazin-1-yl)phenyl)amino)imidazo[1,2-b]pyrimido[4,5-d]pyridazin-5(6H)-one;
6-(2-fluoro-6-methylphenyl)-2-((3-methyl-4-(4-methylpiperazin-1-yl)phenyl)amino)imidazo[1,2-b]pyrimido[4,5-d]pyridazin-5(6H)-one;
6-(2-fluoro-6-methylphenyl)-2-((3,5-dimethyl-4-(4-methylpiperazin-1-yl)phenyl)amino) imidazo[1,2-b]pyrimido[4,5-d]pyridazin-5(6H)-one;
6-(2-chloro-6-methylphenyl)-2-((3-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3,5-dimethyl-4-(4-methylpiperazin-1-yl)phenyl)amino)imidazo[1,2-b]pyrimido[4,5-d]pyridazin-5(6H)-one;
6-(2,6-difluorophenyl)-2-((3-chloro-4-(4-methylpiperazin-1-yl)phenyl)amino)imidazo[1,2-b]pyrimido[4,5-d]pyridazin-5(6H)-one;
6-(2,6-difluorophenyl)-2-((3-fluoro-5-methyl-4-(4-methylpiperazin-1-yl)phenyl)amino)imidazo[1,2-b]pyrimido[4,5-d]pyridazin-5(6)-one;
6-(2,6-difluorophenyl)-2-((3-chloro-5-methyl-4-(4-methylpiperazin-1-yl)phenyl)amino) imidazo[1,2-b]pyrimido[4,5-d]pyridazin-5(6H)-one;
6-(2-chloro-6-fluorophenyl)-2-((3-chloro-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-fluoro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-methyl-4-((3S,5R)-3,4,5-trimethylpiperazin-1-yl)phenyl)amino)imidazo[1,2-b]pyrimido[4,5-d]pyridazin-5(6H)-one;
6-(2-chloro-6-fluorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-methyl-4-(piperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)imidazo[1,2-b]pyrimido[4,5-d]pyridazin-5(6H)-one;
6-(2,6-dichlorophenyl)-2-((3-fluoro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-fluoro-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-chloro-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-methyl-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-methyl-4-((3S,5R)-4-isopropyl-3,5-dimethylpiperazin-1-yl)phenyl)amino)imidazo[1,2-b]pyrimido[4,5-d]pyridazin-5(6H)-one;
6-(2,6-dichlorophenyl)-2-((3-chloro-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-chloro-5-methoxy-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-fluoro-4-(1-methylpiperidin-4-yl)phenyl)amino)imidazo[1,2-b]pyrimido[4,5-d]pyridazin-5(6H)-one;
6-(2,6-dichlorophenyl)-2-((3-chloro-4-(1-methylpiperidin-4-yl)phenyl)amino)imidazo[1,2-b]pyrimido[4,5-d]pyridazin-5(6H)-one;
6-(2,6-dichlorophenyl)-2-((3-methyl-4-(1-methylpiperidin-4-yl)phenyl)amino)imidazo[1,2-b]pyrimido[4,5-d]pyridazin-5(6H)-one;
6-(2,6-dichlorophenyl)-2-((3-fluoro-5-methyl-4-(1-methylpiperidin-4-yl)phenyl)amino)imidazo[1,2-b]pyrimido[4,5-d]pyridazin-5(6H)-one;
6-(2,6-dichlorophenyl)-2-((2,4,4,5-tetramethyl-1,2,3,4-tetrahydroisoquinolin-7-yl)amino)imidazo[1,2-b]pyrimido[4,5-d]pyridazin-5(6H)-one;
6-(2,6-dichlorophenyl)-2-((2,5-dimethyl-1,2,3,4-tetrahydroisoquinolin-7-yl)amino)imidazo[1,2-b]pyrimido[4,5-d]pyridazin-5(6H)-one;
6-(2-bromo-6-fluorophenyl)-2-((3-methyl-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-methyl-4-(4-methylpiperazin-1-yl)phenyl)amino)imidazo[1,2-b]pyrimido[4,5-d]pyridazin-5(6H)-one;
6-(2-bromo-6-chlorophenyl)-2-((3-chloro-4-(4-methylpiperazin-1-yl)phenyl)amino)imidazo[1,2-b]pyrimido[4,5-d]pyridazin-5(6H)-one;
6-(2-bromo-6-chlorophenyl)-2-((3-chloro-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-methyl-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2-fluoro-6-methylphenyl)-2-((3-chloro-4-(4-methylpiperazin-1-yl)phenyl)amino)imidazo[1,2-b]pyrimido[4,5-d]pyridazin-5(6H)-one;
6-(2-fluoro-6-methylphenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3-chloro-4-(4-methylpiperazin-1-yl)phenyl)amino)imidazo[1,2-b]pyrimido[4,5-d]pyridazin-5(6H)-one;
6-(2-chloro-6-methylphenyl)-2-((3-fluoro-5-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)-8-methylimidazo[1,2-b]pyrimido[4,5-d]pyridazin-5(6H)-one;
6-(2,6-dichlorophenyl)-2-((3-chloro-4-(4-methylpiperazin-1-yl)phenyl)amino)-9-methylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-methylimidazo[1,2-b]pyrimido[4,5-d]pyridazin-5(6H)-one;
6-(2,6-dichlorophenyl)-2-((3-fluoro-5-methyl-4-(4-(dimethylamino)piperidin-1-yl)phenyl)amino)-9-methylimidazo[1,2-b]pyrimido[4,5-d]pyridazin-5(6H)-one;
6-(2,6-dichlorophenyl)-2-((3-methyl-4-(4-methylpiperazin-1-yl)phenyl)amino)-9-ethylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-ethylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-isopropylimidazo[1,2-b]pyrimido[4,5-d]pyridazin-5(6H)-one;
6-(2,6-dichlorophenyl)-2-((3-methyl-4-(4-methylpiperazin-1-yl)phenyl)amino)-9-(hydroxymethyl)imidazo[1,2-b]pyrimido[4,5-d]pyridazin-5(6H)-one;
6-allyl-2-((3-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((4-((3S,5R)-3,5-dimethylpiperazin-1-yl)-3-methylphenyl)amino)-8,9-dihydroimidazo[1,2-a]pyrimido[5,4-e]pyrimidin-5(6H)-one;
6-(2,6-dichlorophenyl)-2-((4-((3S,5R)-3,5-dimethyl-4-(methyl-d3)piperazin-1-yl)-3-methylphenyl)amino)-8,9-dihydroimidazo[1,2-a]pyrimido[5,4-e]pyrimidin-5(6H)-one;
2-((3-bromo-4-((3S,5R)-3,4,5-trimethylpiperazin-1-yl)phenyl)amino)-6-(2,6-dichlorophenyl)-8,9-dihydroimidazo[1,2-a]pyrimido[5,4-e]pyrimidin-5(6H)-one;
2-((3-bromo-5-methyl-4-((3S,5R)-3,4,5-trimethylpiperazin-1-yl)phenyl)amino)-6-(2,6-dichlorophenyl)-8,9-dihydroimidazo[1,2-a]pyrimido[5,4-e]pyrimidin-5(6H)-one;
6-(2,6-dichlorophenyl)-2-((3-fluoro-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dimethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5S)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(piperidin-4-yl)phenyl)amino)-8,9-dihydroimidazo[1,2-a]pyrimido[5,4-e]pyrimidin-5(6H)-one;
6-(2,6-dichlorophenyl)-2-((3-fluoro-4-(1-methylpiperidin-4-yl)phenyl)amino)-8,9-dihydroimidazo[1,2-a]pyrimido[5,4-e]pyrimidin-5(6H)-one;
2-((3-chloro-4-(1-methylpiperidin-4-yl)phenyl)amino)-6-(2,6-dichlorophenyl)-8,9-dihydroimidazo[1,2-a]pyrimido[5,4-e]pyrimidin-5(6H)-one;
6-(2,6-dichlorophenyl)-2-((3-methyl-4-(1-methylpiperidin-4-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(1-methylpiperidin-4-yl)phenyl)amino)-8,9-dihydroimidazo[1,2-a]pyrimido[5,4-e]pyrimidin-5(6H)-one;
or a pharmaceutically acceptable salt or prodrug thereof.

20. The method of claim 9, wherein the Wee1 kinase inhibitor is 6-(2,6-dichlorophenyl)-2-((3-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one, or a pharmaceutically acceptable salt or prodrug thereof.

21. The method of claim 9, wherein the cancer is selected from the group consisting of: diffuse midline glioma, childhood diffuse pontine glioma, midline glioma, liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, lung cancer, Wilms tumor, cervical cancer, testicular cancer, soft tissue sarcoma, chronic lymphocytic leukemia, primary macroglobulinemia, bladder cancer, chronic myeloid leukemia, primary brain cancer, malignant melanoma, small cell lung cancer, gastric cancer, colon cancer, malignant pancreatic islet tumor, malignant carcinoid cancer, malignant melanoma, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer; preferably, the cancer is selected from diffuse midline glioma, childhood diffuse pontine glioma and midline glioma.

22. The method of claim 12, wherein the anti-cancer drug is selected from the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, imatinib, gefitinib, erlotinib, osimertinib, afatinib, ceritinib, alectinib, crizotinib, erlotinib, lapatinib, sorafenib, sunitinib, nilotinib, dasatinib, pazopanib, torisel, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, Ixazomib, vismodegib, sonidegib, denosumab, thalidomide, lenalidomide, Venetoclax, Aldesleukin, Sipueucel-T, Palbociclib, Olaparib, Niraparib, Rucaparib, Senaparib and Talazoparib.

23. The method of claim 13, wherein the oligodendrocyte precursor cells of the patient are detected to determine whether they contain the H3K27M mutation, and presence of the H3K27M mutation in the oligodendrocyte precursor cells indicates response of the patient to the Wee1 kinase inhibitor

24. The method of claim 13, wherein the patient is suffered from a cancer selected from the group consisting of: diffuse midline glioma, childhood diffuse pontine glioma, midline glioma, liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, lung cancer, Wilms tumor, cervical cancer, testicular cancer, soft tissue sarcoma, chronic lymphocytic leukemia, primary macroglobulinemia, bladder cancer, chronic myeloid leukemia, primary brain cancer, malignant melanoma, small cell lung cancer, gastric cancer, colon cancer, malignant pancreatic islet tumor, malignant carcinoid cancer, malignant melanoma, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer; preferably, the cancer is selected from diffuse midline glioma, childhood diffuse pontine glioma and midline glioma.

25. The method of claim 13, wherein the Wee1 kinase inhibitor is selected from compounds of Formula I: 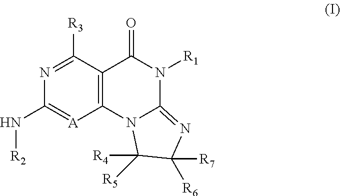
or pharmaceutically acceptable salts or prodrugs thereof, wherein:
A is N or CR₁₅;
R is H, optionally substituted C₁₋₈ alkyl, optionally substituted C₂₋₈ alkenyl, optionally substituted C₃-8 cycloalkyl, optionally substituted aryl, or optionally substituted heteroaryl;
R₂ is an optionally substituted heterocyclic group, an optionally substituted aryl, or an optionally substituted heteroaryl;
R₃-R₇ and R₁₅ are independently H, halo, optionally substituted amino, optionally substituted alkoxy, optionally substituted C₁₋₁₀ alkyl, haloalkyl, alkenyl, alkynyl, hydroxylalkyl, aminoalkyl, carboxylalkyl, nitro, cyano, acylamido, hydroxy, thiol, acyloxy, azido, carboxy, ethylenedioxo, carbonylamido or optionally substituted alkylthiol;
or the Wee1 kinase inhibitor is selected from compounds of Formula II: 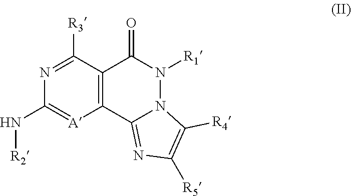
or pharmaceutically acceptable salts or prodrugs thereof, wherein:
A′ is N or CR₆′;
R₁′ is H, optionally substituted C₁-C₈ alkyl, optionally substituted C₂-C₈ alkenyl, optionally substituted C₃-C₈ cycloalkyl, optionally substituted aryl, optionally substituted heterocyclic group or optionally substituted heteroaryl;
R₂′ is an optionally substituted carbocyclic group, an optionally substituted heterocyclic group, an optionally substituted aryl, or an optionally substituted heteroaryl;
R₃′-R₆′ are independently H, halo, optionally substituted amino, optionally substituted alkoxy, optionally substituted C₁-C₁₀ alkyl, alkenyl, alkynyl, nitro, cyano, acylamido, hydroxy, thiol, acyloxy, azido, carboxy, ethylenedioxo, carbonylamido or optionally substituted alkylthiol.

26. The method of claim 25, wherein the optionally substituted C₁-C₁₀ alkyl defined in R₃′-R₆′ is haloalkyl, hydroxylalkyl, aminoalkyl or carboxylalkyl.

27. The method of claim 13, wherein the Wee1 kinase inhibitor is a compound represented by Formula III: 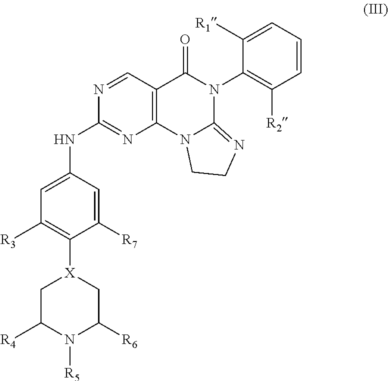
or a stereoisomer thereof, or a pharmaceutically acceptable salt or prodrug thereof, wherein R₁″ and R₂″ are independently halo; R₃″ is halo, C₁₋₄ alkyl or C₁₋₄ alkoxy; R₄″ and R₆″ are each independently H or C₁₋₄ alkyl; R₃″ is H or C₁₋₄ alkyl; R₇″ is H, halo, C₁₋₄ alkyl or C₁₋₄ alkoxy; and X is CH or N;

28. The method of claim 13, wherein the Wee1 kinase inhibitor is selected from the group consisting of:
6-(2-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-8,9-dihydroimidazo[1,2-a]pyrimido[5,4-e]pyrimidin-5(6H)-one;
4-(2-chlorophenyl)-8-((4-(4-methylpiperazin-1-yl)phenyl)amino)-1,2-dihydroimidazo[1,2-a]pyrido[3,4-e]pyrimidin-5(4H)-one;
6-(2,6-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-isopropylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-acetylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2-chloro-6-fluorophenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2-chloro-6-fluorophenyl)-2-((2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2-chloro-6-fluorophenyl)-2-((2′-acetyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dimethylphenyl)-2-((2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dimethylphenyl)-2-((2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dimethylphenyl)-2-((2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-isopropyl-2-((4-(4-methylpiperazin-1-yl)phenyl)amino)-8,9-dihydroimidazo[1,2-a]pyrimido[5,4-e]pyrimidin-5(6H)-one;
6-tert-butyl-2-((4-(4-methylpiperazin-1-yl)phenyl)amino)-8,9-dihydroimidazo[1,2-a]pyrimido[5,4-e]pyrimidin-5(6H)-one;
6-cyclopropyl-2-((4-(4-methylpiperazin-1-yl)phenyl)amino)-8,9-dihydroimidazo[1,2-a]pyrimido[5,4-e]pyrimidin-5(6H)-one;
6-cyclohexyl-2-((4-(4-methylpiperazin-1-yl)phenyl)amino)-8,9-dihydroimidazo[1,2-a]pyrimido[5,4-e]pyrimidin-5(6H)-one;
6-allyl-2-((4-(4-methylpiperazin-1-yl)phenyl)amino)-8,9-dihydroimidazo[1,2-a]pyrimido[5,4-e]pyrimidin-5(6H)-one;
6-(thiophen-2-yl)-2-((4-(4-methylpiperazin-1-yl)phenyl)amino)-8,9-dihydroimidazo[1,2-a]pyrimido[5,4-e]pyrimidin-5(6H)-one;
6-(furan-2-yl)-2-((4-(4-methylpiperazin-1-yl)phenyl)amino)-8,9-dihydroimidazo[1,2-a]pyrimido[5,4-e]pyrimidin-5(6H)-one;
6-(1H-pyrrol-2-yl)-2-((4-(4-methylpiperazin-1-yl)phenyl)amino)-8,9-dihydroimidazo[1,2-a]pyrimido[5,4-e]pyrimidin-5(6H)-one;
6-(1H-imidazol-5-yl)-2-((4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-8,8-dimethyl-8,9-dihydroimidazo[1,2-a]pyrimido[5,4-e]pyrimidin-5(6H)-one;
6-(2-chloro-6-fluorophenyl)-2-((4-(4-methylpiperazin-1-yl)phenyl)amino)-9,9-dimethyl-8,9-dihydroimidazo[1,2-a]pyrimido[5,4-e]pyrimidin-5(6H)-one;
6-(2-chloro-6-fluorophenyl)-2-((4-((2S,6R)-2,6-dimethylmorpholino)phenyl)amino)-8,9-dihydroimidazo[1,2-a]pyrimido[5,4-e]pyrimidin-5(6H)-one;
6-(2-chloro-6-fluorophenyl)-2-((4-(morpholinomethyl)phenyl)amino)-8,9-dihydroimidazo[1,2-a]pyrimido[5,4-e]pyrimidin-5(6H)-one;
6-(2-chloro-6-fluorophenyl)-2-((4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-fluoro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-fluoro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-chloro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-2-(trifluoromethyl)phenyl)amino)-8,9-dihydroimidazo[1,2-a]pyrimido[5,4-e]pyrimidin-5(6H)-one;
6-(2-chloro-6-fluorophenyl)-2-((2-trifluoromethyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2-methyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-3-(trifluoromethyl)phenyl)amino)-8,9-dihydroimidazo[1,2-a]pyrimido[5,4-e]pyrimidin-5(6H)-one;
6-(2-chloro-6-fluorophenyl)-2-((3-trifluoromethyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-isopropylpiperazin-1-yl)-3-methylphenyl)amino)-8,9-dihydroimidazo[1,2-a]pyrimido[5,4-e]pyrimidin-5(6H)-one;
6-(2-chloro-6-fluorophenyl)-2-((4-((3S,5R)-4-isopropyl-3,5-dimethylpiperazin-1-yl)-3-methylphenyl)amino)-8,9-dihydroimidazo[1,2-a]pyrimido[5,4-e]pyrimidin-5(6H)-one;
6-(2-chloro-6-fluorophenyl)-2-((3-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methylpiperazin-1-yl)-3-nitrophenyl)amino)-8,9-dihydroimidazo[1,2-a]pyrimido[5,4-e]pyrimidin-5(6H)-one;
6-(2-chloro-6-fluorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((6-(4-methylpiperazin-1-yl)pyridin-3-yl)amino)-8,9-dihydroimidazo[1,2-a]pyrimido[5,4-e]pyrimidin-5(6H)-one;
6-(2-chloro-6-fluorophenyl)-2-((2′-isopropyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2-chloro-6-fluorophenyl)-2-((5-methyl-4,5,6,7-tetrahydropyrazolo[1,5-a]pyrazin-2-yl)amino)-8,9-dihydroimidazo[1,2-a]pyrimido[5,4-e]pyrimidin-5(6H)-one;
6-(2,6-difluorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-(trifluoromethyl)phenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-isopropylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-((3R,5S)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-((3S,5R)-4-isopropyl-3,5-dimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-chloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((2-trifluoromethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-methylpiperazin-1-yl)-3-(trifluoromethyl)phenyl)amino)-8,9-dihydroimidazo[1,2-a]pyrimido[5,4-e]pyrimidin-5(6H)-one;
6-(2,6-dichlorophenyl)-2-((3-trifluoromethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-isopropylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5R)-4-isopropyl-3,5-dimethylpiperazin-1-yl)-phenyl)amino)-8,9-dihydroimidazo[1,2-a]pyrimido[5,4-e]pyrimidin-5(6H)-one;
6-(2,6-dichlorophenyl)-2-((5-methyl-4,5,6,7-tetrahydropyrazolo[1,5-a]pyrazin-2-yl)amino)-8,9-dihydroimidazo[1,2-a]pyrimido[5,4-e]pyrimidin-5(6H)-one;
6-(2-chloro-6-trifluoromethylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methoxyphenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2,6-dimethylphenyl)-2-((4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(4-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(3-chlorophenyl)-2-((4-(4-methylpiperazin-1-yl)phenyl)amino)-8,9-dihydroimidazo[1,2-a]pyrimido[5,4-e]pyrimidin-5(6H)-one;
6-(2,4-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-4-fluorophenyl)-2-((4-(4-methylpiperazin-1-yl)phenyl)amino)-8,9-dihydroimidazo[1,2-a]pyrimido[5,4-e]pyrimidin-5(6H)-one;
6-(2-chloro-3-fluorophenyl)-2-((4-(4-methylpiperazin-1-yl)phenyl)amino)-8,9-dihydroimidazo[1,2-a]pyrimido[5,4-e]pyrimidin-5(6H)-one;
6-(2,5-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(2,3-dichlorophenyl)-2-((4-(4-methylpiperazin-1-yl)phenyl)amino)-8,9-dihydroimidazo[1,2-a]pyrimido[5,4-e]pyrimidin-5(6H)-one;
6-(pyrimidin-2-yl)-2-((4-(4-methylpiperazin-1-yl)phenyl)amino)-8,9-dihydroimidazo[1,2-a]pyrimido[5,4-e]pyrimidin-5(6H)-one;
2-((4-(4-methylpiperazin-1-yl)phenyl)amino)-8,9-dihydroimidazo[1,2-a]pyrimido[5,4-e]pyrimidin-5(6H)-one;
6-cyclobutyl-2-((4-(4-methylpiperazin-1-yl)phenyl)amino)-8,9-dihydroimidazo[1,2-a]pyrimido[5,4-e]pyrimidin-5(6H)-one;
6-cyclopentyl-2-((4-(4-methylpiperazin-1-yl)phenyl)amino)-8,9-dihydroimidazo[1,2-a]pyrimido[5,4-e]pyrimidin-5(6H)-one;
6-phenyl-2-((4-(4-methylpiperazin-1-yl)phenyl)amino)-8,9-dihydroimidazo[1,2-a]pyrimido[5,4-e]pyrimidin-5(6H)-one;
6-(pyridin-2-yl)-2-((4-(4-methylpiperazin-1-yl)phenyl)amino)-8,9-dihydroimidazo[1,2-a]pyrimido[5,4-e]pyrimidin-5(6H)-one;
6-(pyridin-3-yl)-2-((4-(4-methylpiperazin-1-yl)phenyl)amino)-8,9-dihydroimidazo[1,2-a]pyrimido[5,4-e]pyrimidin-5(6H)-one;
6-(pyridin-4-yl)-2-((4-(4-methylpiperazin-1-yl)phenyl)amino)-8,9-dihydroimidazo[1,2-a]pyrimido[5,4-e]pyrimidin-5(6H)-one;
6-(2,6-difluorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2,6-difluorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((4-(1H-imidazol-1-yl)phenyl)amino)-8,9-dihydroimidazo[1,2-a]pyrimido[5,4-e]pyrimidin-5(6H)-one;
6-(2-chloro-6-fluorophenyl)-2-((4-(4-methyl-1,4-diazecyclohept-1-yl)phenyl)amino)-8,9-dihydroimidazo[1,2-a]pyrimido[5,4-e]pyrimidin-5(6H)-one;
6-(2-chloro-6-fluorophenyl)-2-((4-((4-methylpiperazin-1-yl)methyl)phenyl)amino)-8,9-dihydroimidazo[1,2-a]pyrimido[5,4-e]pyrimidin-5(6H)-one;
6-(2-chloro-6-fluorophenyl)-2-((4-(2-(dimethylamino)ethoxy)phenyl)amino)-8,9-dihydroimidazo[1,2-a]pyrimido[5,4-e]pyrimidin-5(6H)-one;
6-(2-chloro-6-fluorophenyl)-2-((4-(3-(dimethylamino)propoxy)phenyl)amino)-8,9-dihydroimidazo[1,2-a]pyrimido[5,4-e]pyrimidin-5(6H)-one;
6-(2-chloro-6-fluorophenyl)-2-((4-((1-methylpiperidin-4-yl)oxy)phenyl)amino)-8,9-dihydroimidazo[1,2-a]pyrimido[5,4-e]pyrimidin-5(6H)-one;
6-(2-chloro-6-fluorophenyl)-2-((4-((2-(dimethylamino)ethyl)amino)phenyl)amino)-8,9-dihydroimidazo[1,2-a]pyrimido[5,4-e]pyrimidin-5(6H)-one;
6-(2-chloro-6-fluorophenyl)-2-((4-((2-(dimethylamino)ethyl)(methyl)amino)phenyl)amino)-8,9-dihydroimidazo[1,2-a]pyrimido[5,4-e]pyrimidin-5(6H)-one;
6-(2-chloro-6-fluorophenyl)-2-((4-((3-(dimethylamino)propyl)amino)phenyl)amino)-8,9-dihydroimidazo[1,2-a]pyrimido[5,4-e]pyrimidin-5(6H)-one;
6-(2-chloro-6-fluorophenyl)-2-((4-((3-(dimethylamino)propyl)(methyl)amino)phenyl)amino)-8,9-dihydroimidazo[1,2-a]pyrimido[5,4-e]pyrimidin-5(6H)-one;
6-(2-chloro-6-fluorophenyl)-2-((4-((1-methylpiperidin-4-yl)amino)phenyl)amino)-8,9-dihydroimidazo[1,2-a]pyrimido[5,4-e]pyrimidin-5(6H)-one;
6-(2-chloro-6-fluorophenyl)-2-((4-(methyl(1-methylpiperidin-4-yl)amino)phenyl)amino)-8,9-dihydroimidazo[1,2-a]pyrimido[5,4-e]pyrimidin-5(6H)-one;
6-(2-chloro-6-fluorophenyl)-2-((4-(1-methylpiperidin-4-yl)phenyl)amino)-8,9-dihydroimidazo[1,2-a]pyrimido[5,4-e]pyrimidin-5(6H)-one;
6-(2-chloro-6-fluorophenyl)-2-((4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-bromo-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-4-(4-(dimethylamino)piperidin-1-yl)-phenyl)amino)-8,9-dihydroimidazo[1,2-a]pyrimido[5,4-e]pyrimidin-5(6H)-one;
6-(2-chloro-6-fluorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-fluorophenyl)-2-((5-(4-methylpiperazin-1-yl)pyrazin-2-yl)amino)-8,9-dihydroimidazo[1,2-a]pyrimido[5,4-e]pyrimidin-5(6H)-one;
6-(2-chloro-6-fluorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-fluoro-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-chloro-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-bromo-5-methoxy-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methyl-5-trifluoromethyl-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-methoxy-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((5-chloro-2-methyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,5-dimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((5-chloro-2,4,4-trimethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((2,4,4,5-tetramethyl-1,2,3,4-tetrahydroisoquinolin-7-yl)amino)-8,9-dihydroimidazo[1,2-a]pyrimido[5,4-e]pyrimidin-5(6H)-one;
6-(2,6-dichlorophenyl)-2-((5′-chloro-2′-methyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((2′,5′-dimethyl-2′,3′-dihydro-1′H-spiro[cyclopropane-1,4′-isoquinolin]-7′-yl)amino)-8,9-dihydroimidazo[1,2-a]pyrimido[5,4-e]pyrimidin-5(6H)-one;
6-(2,6-dichlorophenyl)-2-((3,5-dichloro-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-bromo-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3,5-dichloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-(dimethylamino)piperidin-1-yl)-5-methoxyphenyl)amino)-8,9-dihydroimidazo[1,2-a]pyrimido[5,4-e]pyrimidin-5(6H)-one;
6-(2,6-dichlorophenyl)-2-((3-bromo-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methoxy-4-(4-methylpiperazin-1-yl)phenyl)amino)-6-(2,6-dichlorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-trifluoromethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-fluoro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-chloro-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-methyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-bromo-5-methoxy-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-bromo-6-chlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-bromo-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-fluoro-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-bromo-6-chlorophenyl)-2-((3-bromo-5-methyl-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-fluoro-6-methylphenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-fluoro-5-methyl-4-(4-(dimethylamino)piperidin-1-yl)phenyl)amino)-8,9-dihydroimidazo[1,2-a]pyrimido[5,4-e]pyrimidin-5(6H)-one;
6-(2-chloro-6-methylphenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3,5-dichloro-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3-chloro-5-methyl-4-(4-methylpiperazin-1-yl)phenyl)amino)-8,9-dihydroimidazo[1,2-a]pyrimido[5,4-e]pyrimidin-5(6H)-one;
6-(2-chloro-6-methylphenyl)-2-((3,5-dimethyl-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-4-(piperazin-1-yl)phenyl)amino)-8,9-dihydroimidazo[1,2-a]pyrimido[5,4-e]pyrimidin-5(6H)-one;
6-(2-chloro-6-fluorophenyl)-2-((3-(hydroxymethyl)-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-(hydroxymethyl)-4-(piperazin-1-yl)phenyl)amino)-8,9-dihydroimidazo[1,2-a]pyrimido[5,4-e]pyrimidin-5(6H)-one;
6-(2,6-dichlorophenyl)-2-((3-(hydroxymethyl)-4-(4-methylpiperazin-1-yl)phenyl)amino)-8,9-dihydroimidazo[1,2-a]pyrimido[5,4-e]pyrimidin-5(6H)-one;
6-(2,6-dichlorophenyl)-2-((4-(4-(2-hydroxyethyl)piperazin-1-yl)phenyl)amino)-8,9-dihydroimidazo[1,2-a]pyrimido[5,4-e]pyrimidin-5(6H)-one;
6-(2,6-dichlorophenyl)-2-((4-morpholinophenyl)amino)-8,9-dihydroimidazo[1,2-a]pyrimido[5,4-e]pyrimidin-5(6H)-one;
6-(2,6-dichlorophenyl)-2-((3-((methylamino)methyl)-4-morpholinophenyl)amino)-8,9-dihydroimidazo[1,2-a]pyrimido[5,4-e]pyrimidin-5(6H)-one;
6-(2-bromo-6-chlorophenyl)-2-((3-chloro-4-(4-(dimethylamino)piperidin-1-yl)phenyl)amino)-8,9-dihydroimidazo[1,2-a]pyrimido[5,4-e]pyrimidin-5(6H)-one;
6-(2,6-dichlorophenyl)-2-((3-chloro-4-(4-methylpiperazin-1-yl)phenyl)amino)-8,9-dihydroimidazo[1,2-a]pyrimido[5,4-e]pyrimidin-5(6H)-one;
6-(2-chloro-6-fluorophenyl)-2-((2,4,4-trimethyl-1,2,3,4-tetrahydroisoquinolin-7-yl)amino)imidazo[1,2-b]pyrimido[4,5-d]pyridazin-5(6H)-one;
6-(2,6-difluorophenyl)-2-((3-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2,6-dichlorophenyl)-2-((3,5-dimethyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3,5-dimethyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((2,4,4-trimethyl-1,2,3,4-tetrahydroisoquinolin-7-yl)amino)imidazo[1,2-b]pyrimido[4,5-d]pyridazin-5(6H)-one;
6-(2,6-difluorophenyl)-2-((3,5-dimethyl-4-(4-methylpiperazin-1-yl)phenyl)amino)imidazo[1,2-b]pyrimido[4,5-d]pyridazin-5(6H)-one;
6-(2-fluoro-6-methylphenyl)-2-((3-methyl-4-(4-methylpiperazin-1-yl)phenyl)amino)imidazo[1,2-b]pyrimido[4,5-d]pyridazin-5(6H)-one;
6-(2-fluoro-6-methylphenyl)-2-((3,5-dimethyl-4-(4-methylpiperazin-1-yl)phenyl)amino) imidazo[1,2-b]pyrimido[4,5-d]pyridazin-5(6H)-one;
6-(2-chloro-6-methylphenyl)-2-((3-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3,5-dimethyl-4-(4-methylpiperazin-1-yl)phenyl)amino)imidazo[1,2-b]pyrimido[4,5-d]pyridazin-5(6H)-one;
6-(2,6-difluorophenyl)-2-((3-chloro-4-(4-methylpiperazin-1-yl)phenyl)amino)imidazo[1,2-b]pyrimido[4,5-d]pyridazin-5(6H)-one;
6-(2,6-difluorophenyl)-2-((3-fluoro-5-methyl-4-(4-methylpiperazin-1-6-(2,6-difluorophenyl)-2-((3-chloro-5-methyl-4-(4-methylpiperazin-1-yl)phenyl)amino) imidazo[1,2-b]pyrimido[4,5-d]pyridazin-5(6H)-one;
6-(2-chloro-6-fluorophenyl)-2-((3-chloro-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-fluoro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-fluorophenyl)-2-((3-methyl-4-((3S,5R)-3,4,5-trimethylpiperazin-1-yl)phenyl)amino)imidazo[1,2-b]pyrimido[4,5-d]pyridazin-5(6H)-one;
6-(2-chloro-6-fluorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-methyl-4-(piperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)imidazo[1,2-b]pyrimido[4,5-d]pyridazin-5(6H)-one;
6-(2,6-dichlorophenyl)-2-((3-fluoro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-fluoro-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-chloro-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-methyl-4-((3S,5R)-3,4,5-trimethylpiperazin-1-yl)phenyl)amino)imidazo[1,2-b]pyrimido[4,5-d]pyridazin-5(6H)-one;
6-(2,6-dichlorophenyl)-2-((3-methyl-4-((3S,5R)-4-isopropyl-3,5-dimethylpiperazin-1-yl)phenyl)amino)imidazo[1,2-b]pyrimido[4,5-d]pyridazin-5(6H)-one;
6-(2,6-dichlorophenyl)-2-((3-chloro-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-chloro-5-methoxy-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((3-fluoro-4-(1-methylpiperidin-4-yl)phenyl)amino)imidazo[1,2-b]pyrimido[4,5-d]pyridazin-5(6H)-one;
6-(2,6-dichlorophenyl)-2-((3-chloro-4-(1-methylpiperidin-4-yl)phenyl)amino)imidazo[1,2-b]pyrimido[4,5-d]pyridazin-5(6H)-one;
6-(2,6-dichlorophenyl)-2-((3-methyl-4-(1-methylpiperidin-4-yl)phenyl)amino)imidazo[1,2-b]pyrimido[4,5-d]pyridazin-5(6H)-one;
6-(2,6-dichlorophenyl)-2-((3-fluoro-5-methyl-4-(1-methylpiperidin-4-yl)phenyl)amino)imidazo[1,2-b]pyrimido[4,5-d]pyridazin-5(6H)-one;
6-(2,6-dichlorophenyl)-2-((2,4,4,5-tetramethyl-1,2,3,4-tetrahydroisoquinolin-7-yl)amino)imidazo[1,2-b]pyrimido[4,5-d]pyridazin-5(6H)-one;
6-(2,6-dichlorophenyl)-2-((2,5-dimethyl-1,2,3,4-tetrahydroisoquinolin-7-yl)amino)imidazo[1,2-b]pyrimido[4,5-d]pyridazin-5(6H)-one;
6-(2-bromo-6-fluorophenyl)-2-((3-methyl-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-methyl-4-(4-methylpiperazin-1-yl)phenyl)amino)imidazo[1,2-b]pyrimido[4,5-d]pyridazin-5(6H)-one;
6-(2-bromo-6-chlorophenyl)-2-((3-chloro-4-(4-methylpiperazin-1-yl)phenyl)amino)imidazo[1,2-b]pyrimido[4,5-d]pyridazin-5(6H)-one;
6-(2-bromo-6-chlorophenyl)-2-((3-chloro-4-(4-(dimethylamino)piperidin-1-yl)phenyl)amino)imidazo[1,2-b]pyrimido[4,5-d]pyridazin-5-(6H)-one;
6-(2-bromo-6-chlorophenyl)-2-((3-methyl-4-(4-(dimethylamino)piperidin-1-yl)phenyl)amino)imidazo[1,2-b]pyrimido[4,5-d]pyridazin-5(6H)-one;
6-(2-bromo-6-chlorophenyl)-2-((3-fluoro-5-methyl-4-(4-(dimethylamino)piperidin-1-yl)phenyl)amino)imidazo[1,2-b]pyrimido[4,5-d]pyridazin-5(6H)-one;
6-(2-fluoro-6-methylphenyl)-2-((3-chloro-4-(4-methylpiperazin-1-yl)phenyl)amino)imidazo[1,2-b]pyrimido[4,5-d]pyridazin-5(6H)-one;
6-(2-fluoro-6-methylphenyl)-2-((3-chloro-5-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3-chloro-4-(4-methylpiperazin-1-yl)phenyl)amino)imidazo[1,2-b]pyrimido[4,5-d]pyridazin-5(6H)-one;
6-(2-chloro-6-methylphenyl)-2-((3-fluoro-5-methyl-4-(4-methylpiperazin-1-yl)phenyl)amino)imidazo[1,2-b]pyrimido[4,5-d]pyridazin-5(6H)-one;
6-(2-chloro-6-methylphenyl)-2-((3-chloro-5-methyl-4-(4-methylpiperazin-1-yl)phenyl)amino)imidazo[1,2-b]pyrimido[4,5-d]pyridazin-5(6H)-one;
6-(2,6-dichlorophenyl)-2-((3-chloro-4-(4-methylpiperazin-1-yl)phenyl)amino)-8-methylimidazo[1,2-b]pyrimido[4,5-d]pyridazin-5(6H)-one;
6-(2,6-dichlorophenyl)-2-((3-chloro-4-(4-methylpiperazin-1-yl)phenyl)amino)-9-methylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-methylimidazo[1,2-b]pyrimido[4,5-d]pyridazin-5(6H)-one;
6-(2,6-dichlorophenyl)-2-((3-fluoro-5-methyl-4-(4-(dimethylamino)piperidin-1-yl)phenyl)amino)-9-methylimidazo[1,2-b]pyrimido[4,5-d]pyridazin-5(6H)-one;
6-(2,6-dichlorophenyl)-2-((3-methyl-4-(4-methylpiperazin-1-yl)phenyl)amino)-9-ethylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-ethylimidazo[1,2-b]pyrimido[4,5-d]pyridazin-5(6H)-one;
6-(2,6-dichlorophenyl)-2-((3-methyl-4-(4-(dimethylamino)piperidin-1-yl)phenyl)amino)-9-isopropylimidazo[1,2-b]pyrimido[4,5-d]pyridazin-5(6H)-one;
6-(2,6-dichlorophenyl)-2-((3-methyl-4-(4-methylpiperazin-1-yl)phenyl)amino)-9-(hydroxymethyl)imidazo[1,2-b]pyrimido[4,5-d]pyridazin-5(6H)-one;
6-allyl-2-((3-methyl-4-(4-(dimethylamino)piperidin-1-yl)phenyl)amino)imidazo[1,2-b]pyrimido[4,5-d]pyridazin-5(6H)-one;
6-(2,6-dichlorophenyl)-2-((4-((3S,5R)-3,5-dimethylpiperazin-1-yl)-3-methylphenyl)amino)-8,9-dihydroimidazo[1,2-a]pyrimido[5,4-e]pyrimidin-5(6H)-one;
6-(2,6-dichlorophenyl)-2-((4-((3S,5R)-3,5-dimethyl-4-(methyl-d3)piperazin-1-yl)-3-methylphenyl)amino)-8,9-dihydroimidazo[1,2-a]pyrimido[5,4-e]pyrimidin-5(6H)-one;
2-((3-bromo-4-((3S,5R)-3,4,5-trimethylpiperazin-1-yl)phenyl)amino)-6-(2,6-dichlorophenyl)-8,9-dihydroimidazo[1,2-a]pyrimido[5,4-e]pyrimidin-5(6H)-one;
2-((3-bromo-5-methyl-4-((3S,5R)-3,4,5-trimethylpiperazin-1-yl)phenyl)amino)-6-(2,6-dichlorophenyl)-8,9-dihydroimidazo[1,2-a]pyrimido[5,4-e]pyrimidin-5(6H)-one;
6-(2,6-dichlorophenyl)-2-((3-fluoro-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3,5-dimethyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methoxy-5-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-((3S,5S)-3,4,5-trimethylpiperazin-1-yl)phenyl)amino)-8,9-dihydroimidazo[1,2-a]pyrimido[5,4-e]pyrimidin-5(6H)-one;
6-(2,6-dichlorophenyl)-2-((3-methyl-4-(piperidin-4-yl)phenyl)amino)-8,9-dihydroimidazo[1,2-a]pyrimido[5,4-e]pyrimidin-5(6H)-one;
6-(2,6-dichlorophenyl)-2-((3-fluoro-4-(1-methylpiperidin-4-yl)phenyl)amino)-8,9-dihydroimidazo[1,2-a]pyrimido[5,4-e]pyrimidin-5(6H)-one;
2-((3-chloro-4-(1-methylpiperidin-4-yl)phenyl)amino)-6-(2,6-dichlorophenyl)-8,9-dihydroimidazo[1,2-a]pyrimido[5,4-e]pyrimidin-5(6H)-one;
6-(2,6-dichlorophenyl)-2-((3-methyl-4-(1-methylpiperidin-4-yl)phenyl)amino)-8,9-dihydroimidazo[1,2-a]pyrimido[5,4-e]pyrimidin-5(6H)-one;
6-(2,6-dichlorophenyl)-2-((3-fluoro-5-methyl-4-(1-methylpiperidin-4-yl)phenyl)amino)-8,9-dihydroimidazo[1,2-a]pyrimido[5,4-e]pyrimidin-5(6H)-one;
or a pharmaceutically acceptable salt or prodrug thereof.

29. The method of claim 13, wherein the Wee1 kinase inhibitor is 6-(2,6-dichlorophenyl)-2-((3-methyl-4-((3S,5R)-3,4,5-trimethylpiperazin-1-yl)phenyl)amino)-8,9-dihydroimidazo[1,2-a]pyrimido[5,4-e]pyrimidin-5(6H)-one, or a pharmaceutically acceptable salt or prodrug thereof.
